SEQUENCE LISTING (12 sequences)

SEQ ID NO: 1 - protein, 357 aa, Gallus gallus
MSGMASDCNSSSQEEYLPHYVQQEDPFASKLSREADIIAGFYLTVIGILSTLGNGYVIFMSSKRKKKLRPAEIMTVNLAVCDLGISVVGKPFSIISFFSHRWIFGWMGCRWYGWAGFFFGCGSLITMTAVSLDRYLKICHLAYGTWLKRHHAFICLALIWAYATFWATVPFAGVGSYAPEPFGTSCTLDWWLAQASVAGQAFVLSILFFCLLFPTAVIVFSYVKIILKVKSSTKEVAHYDTRIQNSHILEMKLTKVAMLICAGFLIAWIPYAVVSVWSAFGQPDSVPIQFSVVPTLLAKSAAMYNPIIYQVIDCKFACCRSGGPKTLQKKSSLKESRMYTLSSHRDSAALSGTQLEV

SEQ ID NO: 2 - protein, 349 aa, Chelonia mydas
MASNHSTSPQDDHLPHYLQDEDPFASKLSREADIVAGFYLTIIGVLSTFGNGYVLFMSLKRKKKLKPAEIMTVNLAMCDLGISVVGKPFSIISFFSHRWVFGWLGCRWYGWAGFFFGCGSLITLTAVSLDRYLKICHLTYGTWLKRHHAFICLAIIWAYATFWATVPFAGLGNYAPEPFGTSCTLDWWLAQGSVAGQAFILNILFFCLLLPTAVIVFSYVKIIAKVKSSAKEVAHYDTRIQNSHVLEIKLTKVAMLICAGFLIAWIPYAVVSVWSAFGQPDSVPIQVSVIPTLLAKSAAMYNPIIYQVIDCKFSCCRSLQKKSSLKKSRLYTISGNREPTEMNETHLEL

SEQ ID NO: 3 - DNA, 89 bp, synthetic construct
  source 1..89: mol_type = other DNA
cgtgaggtaccggatcctctagaatgggcaagcccatccccaaccccctgctgggcctggacagcaccatgagtgggatggcatcggac

SEQ ID NO: 4 - DNA, 47 bp, synthetic construct
  source 1..47: mol_type = other DNA
tcgataagcttgatatcgaattcttagacttccagttgggttccgct

SEQ ID NO: 5 - DNA, 50 bp, synthetic construct
  source 1..50: mol_type = other DNA
tagagtcgagctcaagcttgccaccatgagtgggatggcatcggactgca

SEQ ID NO: 6 - DNA, 50 bp, synthetic construct
  source 1..50: mol_type = other DNA
aaccgcgggccctctagagcatatgttacttgtacagctcgtccatgccg

SEQ ID NO: 7 - DNA, 50 bp, synthetic construct
  source 1..50: mol_type = other DNA
acctccgctgctcgcggggtctagaatgagtgggatggcatcggactgca

SEQ ID NO: 8 - DNA, 50 bp, synthetic construct
  source 1..50: mol_type = other DNA
tatcgataagcttgatatcgaattcttacttgtacagctcgtccatgccg

SEQ ID NO: 9 - DNA, 51 bp, synthetic construct
  source 1..51: mol_type = other DNA
tacattatacgaagttatggcgcgccttattacttgtacagctcgtccatg

SEQ ID NO: 10 - DNA, 52 bp, synthetic construct
  source 1..52: mol_type = other DNA
atactttatacgaagttatgctagccaccatgagtgggatggcatcggactg

SEQ ID NO: 11 - DNA, 50 bp, synthetic construct
  source 1..50: mol_type = other DNA
gcatcacctccgctgctcgcggggtatgagtgggatggcatcggactgca

SEQ ID NO: 12 - DNA, 50 bp, synthetic construct
  source 1..50: mol_type = other DNA
tcaccatggtggcgaccgggggatctgggccaggattctcctcgacgtca

(19) United States
(12) Patent Application Publication
(10) Pub. No.: US 2025/0032577 A1
(43) Pub. Date: Jan. 30, 2025

(54) OPTOGENETIC VISUAL RESTORATION USING LIGHT-SENSITIVE GQ-COUPLED NEUROPSIN (OPSIN 5)

(71) Applicant: GENANS BIOTECHNOLOGY CO., LTD, Changping District Beijing (CN)

(72) Inventors: Tao Yu, Changping District Beijing (CN); Ruicheng Dai, Changping District Beijing (CN); Danwei Weng, Changping District Beijing (CN); Minmin Luo, Changping District Beijing (CN)

(21) Appl. No.: 18/709,350

(22) PCT Filed: Dec. 20, 2022
(86) PCT No.: PCT/CN2022/140490

§ 371 Date: May 10, 2024

(30) Foreign Application Priority Data
Dec. 20, 2021 (WO) PCT/CN2021/139750

(51) Int. Cl. A61K 38/17; A61K 41/00; A61K 48/00; A61P 27/02; C12N 15/86

(52) U.S. Cl. CPC A61K 38/177; A61K 41/008; A61K 48/0033; A61P 27/02; C12N 15/86; C12N 2750/14143

(57) ABSTRACT

Provided is an isolated light-sensitive opsin for rapidly, reversibly, and precisely restoring sensitivity to light of the retinal cell through activating Gq signaling.

### REFERENCE TO SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Apr. 11, 2024, is entitled “WO11698BSUS.xml”, and has a file size of 12,595 bytes.

### INTRODUCTION

G-protein-coupled receptors (GPCRs) modulate many intracellular signaling pathways and represent some of the most intensively studied drug targets (Hauser et al., 2017). Upon ligand binding, the GPCR undergoes a conformation change that is transmitted to heterotrimeric G proteins, which are multi-subunit complexes comprising G_{α} and tightly associated G_{βγ} subunits. The G_{q} proteins, a subfamily of heterotrimeric G_{α} subunits, couple to a class of GPCRs to mediate cellular responses to neurotransmitters, sensory stimuli, and hormones throughout the body. Their primary downstream signaling targets include phospholipase C beta (PLC-β) enzymes, which catalyze the hydrolysis of phospholipid phosphatidylinositol bisphosphate (PIP₂) into inositol trisphosphate (IP₃) and diacylglycerol (DAG). IP₃ triggers the release of Ca²⁺ from intracellular stores into the cytoplasm, and Ca²⁺ together with DAG activate protein kinase C (PKC). Several tools, including chemogenetics and photoactivatable small molecules, have been developed to study the signaling mechanisms and physiological functions of G_{q}-coupled GPCRs and intracellular Ca2+ release.

Optogenetics uses light-responsive proteins to achieve optically-controlled perturbation of cellular activities with genetic specificity and high spatiotemporal precision. Since the early discoveries of optogenetic tools using light-sensitive ion channels and transporters, diverse technologies have been developed and now support optical interventions into intracellular second messengers, protein interactions and degradation, and gene transcription. Opto-a1AR, a creatively designed G_{q}-coupled rhodopsin-GPCR chimera, can induce intracellular Ca²⁺ increase in response to long-time photostimulation (60 s) (Airan et al., 2009). However, this tool has not been widely used, possibly because of its limitations associated with light sensitivity and response kinetics (Tichy et al., 2019). Most animals detect light using GPCR-based photoreceptors, which comprise both a protein moiety (opsin) and a vitamin A derivative (retinal) that functions as both a ligand and a chromophore. Several thousand opsins have been identified to date. Two recent studies, having reported Gᵢ-based opsins from mosquito and lamprey for presynaptic terminals inhibition in neurons, elegantly demonstrated that some naturally occurring photoreceptors are suitable for use as efficient optogenetic tools. Regarding the G_{q} signaling, melanopsin (Opn4) in a subset of mammalian retinal ganglion cells is a G_{q}-coupled opsin that mediates no-image-forming visual functions. However, HEK293 or Neuro-2a cells heterologously expressing Opn4 showed weak light responses and required additional retinal in the culture medium. Opn5 (neuropsin) and its orthologs in many vertebrates have been reported as an ultraviolet (UV)-sensitive opsin that couples to Gᵢ proteins.

Ideal optogenetic tools are urgently needed so as to recover visual function for blind patients.

### SUMMARY OF THE INVENTION

The present invention relates to an isolated light-sensitive opsin for restoring sensitivity to light of the retinal cell through activating G_{q} signaling. The isolated light-sensitive opsin may be used to treat a subject suffering from damage of the external layer of the retina, photoreceptor loss or degeneration, retinal degenerative disease, loss sensitivity to light, or loss light perception, loss of vision, or blindness.

In the first place, the present invention relates to an isolated light-sensitive opsin for restoring sensitivity to light of the retinal cell through activating G_{q} signaling.

In some embodiments, the light has a wavelength ranging range of 360 nm-520 nm, preferably, 450-500, more preferably, 460-480 nm, in particular, 470 nm.

In some embodiments, the isolated opsin is an isolated opsin from an organism, its homologs, its orthologs, its paralogs, fragments or variants thereof having the activity of restoring sensitivity to light of the retinal cell through activating G_{q} signaling.

In some embodiments, the isolated opsin shares at least 70%, at least 80%, at least 90%, at least 95%, at least 96%, at least 97%, at least 98%, or at least 99% identity to the wild type opsin in the organism, its homologs, its orthologs, its paralogs, fragments or variants thereof, and has the activity of restoring sensitivity to light of the retinal cell through activating G_{q} signaling.

In some embodiments, the organism is an animal.

In some embodiments, the isolated opsin is an isolated opsin 5 (Opn5) from an animal, its homologs, its orthologs, its paralogs, fragments or variants thereof having the activity of restoring sensitivity to light of the retinal cell through activating G_{q} signaling.

In some embodiments, the isolated opsin 5 (Opn5) shares at least 70%, at least 80%, at least 90%, at least 95%, at least 96%, at least 97%, at least 98%, or at least 99% identity to the wild type opsin 5 (Opn5) in the animal, its homologs, its orthologs, its paralogs, fragments or variants thereof, and has the activity of restoring sensitivity to light of the retinal cell through activating G_{q} signaling.

In some embodiments, the animal is a vertebrate animal.

In some embodiments, the animal is an avian, a reptile, or a fish, an amphibian, or a mammal.

In some embodiments, the animal is an avian, including but not limited to chicken, duck, goose, ostrich, emu, rhea, kiwi, cassowary, turkey, quail, chicken, falcon, eagle, hawk, pigeon, parakeet, cockatoo, macaw, parrot, perching bird (such as, song bird), jay, blackbird, finch, warbler and sparrow.

In some embodiments, the animal is a reptile including but not limited to lizard, snake, alligator, turtle, crocodile, and tortoise.

In some embodiments, the animal is a fish including but not limited to catfish, eels, sharks, and swordfish.

In some embodiments, the animal is an amphibian including but not limited to a toad, frog, newt, and salamander.

In some embodiments, the isolated opsin 5 (Opn5) is an isolated wild type opsin 5 (Opn5) from the chicken, or fragments or variants thereof having the activity of restoring sensitivity to light of the retinal cell through activating Gq signaling.

In some embodiments, the isolated opsin 5 (Opn5) shares at least 70%, at least 80%, at least 90%, at least 95%, at least 96%, at least 97%, at least 98%, or at least 99% identity to the wild type opsin 5 (Opn5) from the chicken, and has the activity of restoring sensitivity to light of the retinal cell through activating Gq signaling.

In some embodiments, the isolated opsin 5 (Opn5) is an isolated wild type opsin 5 (Opn5) from the turtle, or fragments or variants thereof having the activity of restoring sensitivity to light of the retinal cell through activating G_{q} signaling.

In some embodiments, the isolated opsin 5 (Opn5) shares at least 70%, at least 80%, at least 90%, at least 95%, at least 96%, at least 97%, at least 98%, or at least 99% identity to the wild type opsin 5 (Opn5) from the turtle, and has the activity of restoring sensitivity to light of the retinal cell through activating G_{q} signaling.

In some embodiments, the isolated opsin 5 (Opn5) has the amino acid sequence shown by SEQ ID NO:1 (cOpn5), or fragments or variants thereof having the activity of restoring sensitivity to light of the retinal cell through activating G_{q} signaling.

In some embodiments, the isolated opsin 5 (Opn5) shares at least 70%, at least 80%, at least 90%, at least 95%, at least 96%, at least 97%, at least 98%, or at least 99% identity to the amino acid sequence shown by SEQ ID NO:1 (cOpn5), and has the activity of restoring sensitivity to light of the retinal cell through activating G_{q} signaling.

In some embodiments, the isolated opsin 5 (Opn5) has the amino acid sequence shown by SEQ ID NO:2 (tOpn5), or fragments or variants thereof having the activity of restoring sensitivity to light of the retinal cell through activating Gq signaling.

In some embodiments, the isolated opsin 5 (Opn5) shares at least 70%, at least 80%, at least 90%, at least 95%, at least 96%, at least 97%, at least 98%, or at least 99% identity to the amino acid sequence shown by SEQ ID NO:2 (tOpn5), and has the activity of restoring sensitivity to light of the retinal cell through activating Gq signaling.

The isolated opsin 5 (Opn5) may be used as a convenient optogenetic tool that precisely activates intracellular G_{q} signaling in a retinal cell.

The retinal cell may be a photoreceptor cell, a retinal rod cell, a retinal cone cell, a retinal ganglion cell, a bipolar cell, a ganglion cell, a horizontal cell, a multipolar neuron, a Müller cell, an Amacrine cell, or a Methylnitrosourea.

In the second place, the present invention relates to an isolated nucleic acid encoding the isolated opsin in the first place.

In some embodiments, the isolated nucleic acid encodes the wild type opsin in the organism, its homologs, its orthologs, its paralogs, fragments or variants thereof having the activity of restoring sensitivity to light of the retinal cell through activating G_{q} signaling.

In the third place, the present invention relates to a chimeric gene comprising the sequence of the isolated nucleic acid in the second place operably linked to suitable regulatory sequences.

The chimeric gene further comprises a gene encoding a marker, for example, a fluorescent protein.

In the fourth place, the present invention relates to a vector comprising the isolated nucleic acid in the second place, or the chimeric gene in the third place.

The vector is a eukaryotic vector, a prokaryotic expression vector, a viral vector, or a yeast vector.

In some embodiments, the vector is a herpes virus simplex vector, a vaccinia virus vector, or an adenoviral vector, an adeno-associated viral vector, a retroviral vector, or an insect vector.

Preferably, the vector is a recombinant AAV1, AAV2, AAV3, AAV4, AAV5, AAV6, AAV7, AAV8, AAV9, AAVS, AAVO or AAV10.

In some embodiments, the vector is an expression vector.

In some embodiments, the vector is a gene therapy vector.

In the fifth place, the present invention relates to an isolated cell or a cell culture, comprising the isolated nucleic acid in the second place, the chimeric gene in the third place, or the vector in the fourth place.

For example, expressing cOpn5 in HEK 293T cells powerfully mediates blue light-triggered, G_{q}-dependent Ca²⁺ increase from intracellular stores.

For example, optogenetic activation of cOpn5-expressing astrocytes induces massive ATP release in the mouse brain.

In the sixth place, the present invention relates to use of the isolated opsin in the first place, the isolated nucleic acid in the second place, the chimeric gene in the third place, the vector in the fourth place, or the isolated cell or the cell culture in the fifth place for treating or preventing a disease or a condition mediated by, or involving loss sensitivity to light of the retinal cell.

cOpn5 can be applied to retinal cells and the retinal cells may be activated by light. The light has a wavelength ranging range of 360 nm-520 nm, preferably, 450-500, more preferably, 460-480 nm, in particular, 470 nm.

For example, AAV vector expressing cOpn5-t2a-EGFP is administrated subretinal or intravitreal, and cOpn5 and EGFP are expressed in retinal ganglion cells.

In the seventh place, the present invention relates to a method of treating or preventing a disease or condition mediated by or involving loss sensitivity to light of the retinal cell in a subject, comprising administering the isolated opsin in the first place, the isolated nucleic acid in the second place, the chimeric gene in the third place, the vector in the fourth place, or the isolated cell or the cell culture in the fifth place.

In some embodiments, the disease or condition mediated by or involving loss sensitivity to light of the retinal cell through activating Gq signaling includes but not limited to diseases or conditions benefiting from restoring sensitivity to light of the retinal cell through activating Gq signaling.

In some embodiments, the disease or condition mediated by or involving loss sensitivity to light of the retinal cell through activating Gq signaling includes but not limited to diseases or conditions benefiting from activating retinal cells, such as a photoreceptor cell, a retinal rod cell, a retinal cone cell, a retinal ganglion cell, a bipolar cell, a ganglion cell, a horizontal cell, a multipolar neuron, a Müller cell, an Amacrine cell, or a Methylnitrosourea.

In some embodiments, the disease or condition includes but not limited to damage of the external layer of the retina, photoreceptor loss or degeneration, retinal degenerative disease, loss sensitivity to light, or loss light perception, loss of vision due to a deficit in light perception or sensitivity, or blindness.

In some embodiments, the Opn5 in the present invention may be used to restore sensitivity to light of the retinal cell as long as the retinal ganglion cells are not completely dead.

In some embodiments, the Opn5 in the present invention may be used to treat or prevent diseases associated with degeneration and/or death of retinal ganglion cells (RGC).

In some embodiments, the Opn5 in the present invention may be used to treat or prevent retinitis pigmentosa (RP), macular degeneration, age-related macular degeneration (AMD), autosomal dominant optic atrophy (ADOA), and/or glaucoma.

In some embodiments, the method further comprises applying light having a wavelength range of 360 nm-520 nm, preferably, 450-500 nm, more preferably, 460-480 nm.

In some embodiments, the method further comprises applying two-photon activation using long-wavelength (≥920 nm) light.

The isolated opsin in the present invention is sensitive to the light having a wavelength ranging 360-550 nm, preferably, 450-500, more preferably, 460-480 nm. In particular, 470 nm blue light elicits the strongest Ca²⁺ transients in cells, which means that the isolated opsin in the present invention is ultra-sensitive to the light having a wavelength of 470 nm.

The invention encompasses all combination of the particular embodiments recited herein.

### BRIEF DESCRIPTION OF THE DRAWINGS

FIG. 1 shows that cOpn5 mediates light-induced strong activation of G_{q} signaling in HEK 293T cells.

FIG. 2 shows that cOpn5 couples to G_{q} but not Gᵢ signaling.

FIG. 3 shows that cOpn5 sensitively mediates optical control of G_{q} signaling with high temporal and spatial resolution.

FIG. 4 shows that cOpn5 mediates more rapid and sensitive response to light than opto-a1AR, hM3Dq or opn4.

FIG. 5 shows that cOpn5 effectively mediates the activation of astrocytes.

FIG. 6 shows that health retina contains several cell layers.

FIG. 7 shows that normal mice before MNU-treated have rapid pupillary light response, and C3H/HeNCrl inbred mice do not have pupillary light response.

FIG. 8 shows EGFP in the whole retina after 4 weeks after AAV injection.

FIG. 9 shows that both MNU-treated mice and C3H/HeNCrl mice recover the pupillary light response.

FIG. 10 shows pupillary light response test.

FIG. 11 shows result of immunofluorescence.

FIG. 12 shows result of electrophysiological test.

FIG. 13 shows result of electrophysiological test.

FIG. 14 schematically shows open field avoidance test.

FIG. 15 shows the results of the open field avoidance test.

FIG. 16 shows the restoration of light sensitivity in the eye of the AAV-cOPN5 treated rd1/rd1 mice after 7 weeks (A) and 9 months (B) respectively.

### DESCRIPTION OF PARTICULAR EMBODIMENTS OF THE INVENTION

In the present invention, the capacity of opsin, in particular, Opn5 orthologs from multiple species is tested and it is found that many opsins sensitively and strongly mediated light-induced activation of Gq signaling and/or activating cells. The isolated light-sensitive opsin may be used to treat a subject suffering from damage of the external layer of the retina, photoreceptor loss or degeneration, retinal degenerative disease, loss sensitivity to light, or loss light perception, loss of vision, or blindness.

Preferably, the Opn5 orthologs is chicken ortholog (cOpn5 for simplicity), or turtle ortholog (tOpn5 for simplicity).

Detailed characterizations of Opn5, in particular, cOpn5 reveal that it is super sensitivity to blue light having a wavelength of 450-500 nm, more preferably, 460-480 nm (μW/mm²-level, ˜3 orders of magnitude more sensitive than existing G_{q}-coupled opsin-based tools: opto-a1AR and opn4), high temporal (in response to 10 ms light pulses, ˜3 orders of magnitude more rapidly than opto-a1AR or opn4) and spatial (subcellular level) resolution, and no need of chromophore addition. In particular, endogenous retinal is sufficient and no retinal is needed to be added.

cOpn5 Mediates Optogenetic Activation of G_{q} Signaling and/or Activating Cells.

Specifically, in the present invention, Opn5 orthologs from chicken, turtles, humans and mice (which share 80-90% protein sequence identity from each other) are tested in order to determine whether they have the capacity to mediate blue light-induced Gq signaling activation within HEK 293T cells. Blue light for stimulation and the red intracellular calcium indicator Calbryte™ 630 AM dye are used to monitor the relative Ca²⁺ response. It is found that the Opn5 orthologs from chicken (cOpn5) and turtle (tOpn5) mediated an immediate and strong light-induced increase in Ca²⁺ signal (˜3 ΔF/F), whereas no light effect is observed from cells expressing the human or mouse Opn5 orthologs. As exemplified by the chicken ortholog, the cOpn5 co-localized with the EGFP-CAAX membrane marker, indicating that it is efficiently transported to the plasma membrane. No exogenous retinal is needed to be added to the culture media, which suggests that endogenous retinal is sufficient to render cOpn5 functional. The Ca²⁺ signals are resistant to the removal of extracellular Ca²⁺, thus indicating Ca²⁺ release from the intracellular stores. Preincubation of G_{q} proteins inhibitor, for example, YM-254890, a highly selective G_{q} proteins inhibitor, reversibly abolished the light-induced Ca²⁺ transients in both cOpn5-expressing cells. In cOpn5-, but not human OPN5-expressing cells, a light-induced increase in the level of inositol phosphate (IP1), the rapid degradation product of IP3, is detected; moreover, the extent of this increase is reduced with the treatment of YM-254890. In cOpn5-expressing cells, for example, HEK 293T cells, blue light also triggers the phosphorylation of MARCKS protein, a well-established target of PKC, in a PKC activity-dependent manner. By contrast, blue light illumination effectively reduces cAMP levels in cells expressing human and mouse Opn5 with retinal, but has no such effect in cells expressing cOpn5 without retinal. Collectively, these data support that blue light illumination enables the coupling of cOpn5 to the G_{q} signaling pathway in HEK 293T cells.

cOpn5-Mediated Optogenetics is Sensitive and Precise.

Specifically, the light-activating properties of cOpn5 are characterized in the present invention. cOpn5 may be heterologously expressed in cells, for example, in HEK 293T cells. Although Opn5 is previously considered as an ultraviolet (UV)-sensitive photoreceptor, mapping with a set of wavelengths ranging 365-630 nm at a fixed light intensity of (100 μW/mm²) reveals that the 470 nm blue light elicits the strongest Ca²⁺ transients, with the UVA light (365 and 395 nm) being less effective and longer-wavelength visible light (561 nm or above) completely ineffective. The effects of different light durations on cOpn5-expressing HEK 293T cells are tested, and stimulating with brief light pulses (1, 5, 10, 20, 50 ms; 16 μW/mm2; 470 nm) shows that the Ca²⁺ response achieves the saturation mode with light duration over 10 ms. Longer light durations do not further increase the Ca²⁺ signal amplitude at this light intensity (16 μW/mm²; 470 nm). Delivering 470 nm light at different intensities shows that blue light of ˜4.8 μW/mm² and 16 μW/mm² produce about half maximum and full maximum responses, respectively. These data suggest that the light sensitivity of cOpn5 is 2-3 orders of magnitude higher than the reported values of the commonly used optogenetic tool Channelrhodopsin-2 (ChR2). Together, the results in the present invention indicate that cOpn5 could function as a single-component optogenetic tool without additional retinal, and that cOpn5 is super-sensitive to blue light for its full activation requiring low light intensity (16 μW/mm²) and short duration (10 ms).

The performance of cOpn5 to that of opto-a1AR, a chimera GPCR engineered by mixing rhodopsin with G_{q}-coupled adrenergic receptor is compared. Following the protocol in a previous report, it is found that very long exposure of strong illumination (60 s; 7 mW/mm²) is required to trigger a slow and small (˜0.5 ΔF/F) Ca²⁺ signal increase in opto-a1AR-expressing HEK 293T cells, and 15 s illumination is inefficient. The performance of cOpn5 to that of opn4, a natural opsin which is reported as a tool for Gq signaling activating is compared. It is found that long exposure of strong illumination (25 s; 40 mW/mm²) and additional retinal are required to trigger a slow (˜1 ΔF/F) Ca²⁺ signal increase in opn4-expressing HEK 293T cells. Therefore, compared with existing opsin-based tools (opto-a1AR and opn4), cOpn5 is much more light-sensitive (˜3 orders more sensitive), requires much shorter time exposure (10 ms vs. 60 s), and produces stronger responses.

Furthermore, the performance of cOpn5 to that of the popular G_{q}-coupled chemogenetic tool hM3Dq, which is activated by adding the exogenous small molecule ligand clozapine-N-oxide (CNO) is compared. Light-induced activation of cOpn5-expressing HEK 293T cells has a similar peak response amplitude of the Ca²⁺ signal as CNO-induced activation of hM3Dq-expressing HEK 293T cells. Meanwhile, cOpn5-expressing HEK 293T cells has faster and temporally more precise response, as well as more rapid recovery time than hM3Dq-expressing HEK 293T cells. These results indicate that cOpn5-mediated optogenetics are more controllable in temporal accuracy than those of hM3Dq.

cOpn5 optogenetics allows spatially precise control of cellular activity. Restricting brief light stimulation (63 ms) into a subcellular region of individual cOpn5-expressing HEK 293T cell results in the immediate activation of a single cell. Interestingly, in high cell confluence area, Ca²⁺ signals propagate to surrounding cells, thus suggesting intercellular communication among HEK 293T cells through a yet-to-identified mechanism. cOpn5 is expressed in primary astrocyte cultures prepared from the neonatal mouse brain with AAV vectors for bicistronic expression of cOpn5 and the EGFP marker protein. Using the Calbryte 630 AM dye to monitor Ca²⁺ levels, it is found that blue light illumination of cOpn5-expressing astrocytes produces strong Ca²⁺ transients (˜8 ΔF/F). When the light stimulation (63 ms) is precisely restricted to only subcellular region of an individual cOpn5-expressing astrocyte, it is observed Ca²⁺ signal propagation within the individual cell. Resembling the tests in HEK 293T cells, wave-like propagation of Ca²⁺ signals from the stimulated astrocyte that proceeded gradually to more distal, non-stimulated, astrocytes, is observed. These experiments thus demonstrate that cOpn5 optogenetics allows precise spatial control, and suggest that it may be useful to study the dynamics of astrocytic networks, which was initially discovered using neurochemical and mechanical stimulation.

Here, the present invention demonstrates the use of Opn5 of the present invention as an extremely effective optogenetic tool for restoring sensitivity to light of the retinal cell through activating Gq signaling. Previous studies have characterized mammalian Opn5 as a UV-sensitive Gᵢ-coupled opsin; we present the surprising finding that visible blue light can induce rapid Ca²⁺ transients, IP1 accumulation, and PKC activation in Opn5-expressing, for example cOpn5-expressing or tOpn5-expressing mammalian cells.

Table 6 lists the enabling features of cOpn5 by directly comparing its response amplitudes, light sensitivity, temporal resolution, and the requirement of additional chromophores to those of other optogenetic tools. For cOpn5-expressing cells, merely 10 ms blue light pulses at the intensity of 16 μW/mm² evoke rapid increase in Ca²⁺ signals with the peak amplitudes of 3-8 ΔF/F. By contrast, prior to the present invention, it is revealed that the activation of opto-a1AR or mammalian Opn4, the two proposed optogenetic tools for Gq signaling, require ˜3-fold higher light intensity (7-40 mW/mm²) and prolonged light exposure (20-60 s) and produce only weak Ca²⁺ signals (0.25-0.5 ΔF/F). Therefore, opto-a1AR or mammalian Opn4 cannot mimic the rapid activation profiles of endogenous Gq-coupled receptors that often trigger strong Gq signaling upon subsecond application of their corresponding ligands. By contrast, recent systematic characterizations show that opto-a1AR- and Opn4-mediated optogenetic stimulations do not increase the amplitudes of Ca²⁺ signals and only mildly modulate the frequency of Ca²⁺ transients and synaptic events even after prolonged illumination (Gerasimov et al., 2021; Mederos et al., 2019).

Opn5 in the present invention, in particular, cOpn5 or tOpn5-based optogenetics also enjoys the benefit of safety and convenience. Although Opn5 from many species are reported UV-responsive (Kojima et al., 2011), cOpn5 is optimally activated by 470 nm blue light, which penetrates better than UV and avoids UV-associated cellular toxicity. Its ultra-sensitivity to light also minimizes potential heating artifact. cOpn5 or tOpn5 is strongly, and repetitively activated by light without the requirement for exogenous retinal, possibly because cOpn5 or tOpn5 is a bistable opsin that covalently binds to endogenous retinal and is thus resistant to photo bleaching (Koyanagi and Terakita, 2014; Tsukamoto and Terakita, 2010). By contrast, mammalian experiments of Opn4 requires additional retinal and have long response time and low light sensitivity. Opn5 in the present invention, in particular, cOpn5 or tOpn5 as a single-component system is particularly useful for in vivo studies as it avoids the burden of delivering a compound into the tissue during the experiment.

Opn5 optogenetics in the present invention, in particular, cOpn5 or tOpn5 optogenetics also offers some major advantages over chemogenetics and uncaging tools. It is temporally much more precise and offers single-cell or even subcellular spatial resolution. Opn5 in the present invention, in particular, cOpn5 or tOpn5 also differs from caged compound-based ‘uncaging’ tools such as caged calcium and caged IP3, since these tools require compound preloading and only partially mimic the Ca²⁺-related pathways associated with G_{q} signaling and/or activating cells. There exists other ‘uncaging’ tools, such as caged glutamate and caged ATP (Ellis-Davies, 2007; Lezmy et al., 2021), that target endogenous GPCRs. However, these caged compounds require their introduction into extracellular medium or the intracellular cytoplasm, which limits their applications in behaving animals (Adams and Tsien, 1993b).

Opn5 in the present invention, in particular, cOpn5 or tOpn5, optogenetics should be particularly useful for precisely activating intracellular G_{q} signaling and/or activating cells, which subsequently triggers Ca²⁺ release from intracellular stores and activates PKC. Opn5 in the present invention, in particular, cOpn5 or tOpn5, differs from current channel-based optogenetic tools, such as ChR2 or its variants, which translocate cations across the plasma membrane.

On the basis of the strong light sensitivity of the Opn5 in the present invention, the present invention further demonstrates that the Opn5 in the present invention may be used to restore sensitivity to light of the retinal cell through activating Gq signaling, and thus may be used to treat or alleviate damage of the external layer of the retina, photoreceptor loss or degeneration, retinal degenerative disease, loss sensitivity to light, or loss light perception, loss of vision due to a deficit in light perception or sensitivity, or blindness.

In some embodiments, the Opn5 in the present invention may be used to restore sensitivity to light of the retinal cell as long as the retinal ganglion cells are not completely dead.

In some embodiments, the Opn5 in the present invention may be used to treat or prevent diseases associated with degeneration and/or death of retinal ganglion cells (RGC).

In some embodiments, the Opn5 in the present invention may be used to treat or prevent retinitis pigmentosa (RP), macular degeneration, age-related macular degeneration (AMD), autosomal dominant optic atrophy (ADOA), and/or glaucoma.

In the present invention, cOpn5, cOPN5, O5, and chicken opn5m are used interchangeably.

In the present invention, opn5, OPN5, Opsin and Opn5 are used interchangeably.

The descriptions of particular embodiments and examples are provided by way of illustration and not by way of limitation. Those skilled in the art will readily recognize a variety of noncritical parameters that could be changed or modified to yield essentially similar results.

### EXAMPLES

### Materials and Methods

TABLE 1
Primers for cloning

| | |
|---|---|
| V5-cOpn5 forward primer | 5′-cgtgaggtaccggatcctctagaatgggcaagcccatccccaacc |
| | ccctgctgggcctggacagcaccatgagtgggatggcatcggac-3′ |
| | (SEQ ID NO: 3) |
| V5-cOpn5 reverse primer | 5′-tcgataagcttgatatcgaattcttagacttccagttgggttccgct-3′ |
| | (SEQ ID NO: 4) |
| cOpn5-T2A-eGFP for hSyn promoter | 5′-tagagtcgagctcaagcttgccaccatgagtgggatggcatcggactgca-3′ |
| forward primer | (SEQ ID NO: 5) |
| cOpn5-T2A-eGFP for hSyn promoter | 5′-aaccgcgggccctctagagcatatgttacttgtacagctcgtccatgccg-3′ |
| reverse primer | (SEQ ID NO: 6) |
| cOpn5-T2A-eGFP for GfaABCID | 5′-acctccgctgctcgcggggtctagaatgagtgggatggcatcggactgca-3′ |
| promoter forward primer | (SEQ ID NO: 7) |
| cOpn5-T2A-eGFP for GfaABCID | 5′-tatcgataagcttgatatcgaattcttacttgtacagctcgtccatgccg-3′ |
| promoter reverse primer | (SEQ ID NO: 8) |
| cOpn5-T2A-eGFP for EF1a | 5′-tacattatacgaagttatggcgcgccttattacttgtacagctcgtccatg-3′ |
| promoter forward primer | (SEQ ID NO: 9) |
| cOpn5-T2A-eGFP for EF1a | 5′-atactttatacgaagttatgctagccaccatgagtgggatggcatcggactg-3′ |
| promoter reverse primer | (SEQ ID NO: 10) |
| cOpn5-T2A-mCherry forward primer | 5′-gcatcacctccgctgctcgcggggtatgagtgggatggcatcggactgca-3′ |
| | (SEQ ID NO: 11) |
| cOpn5-T2A-mCherry reverse primer | 5′-tcaccatggtggcgaccgggggatctgggccaggattctcctcgacgtca-3′ |
| | (SEQ ID NO: 12) |

TABLE 2
Recombinant DNA

| | |
|---|---|
| pcDNA3.1-opto-a1AR-EYFP | Addgene plasmid |
| | #20947 |
| EGFP-CAAX | Gift from |
| | Yulong Li |
| pLJM1-EGFP | Addgene plasmid |
| | #19319 |
| pAAV-GfaABC1D-hM3D(Gq)-mCherry | Addgene Plasmid |
| | #50478 |
| pAAV-EF1a-DIO-eGFP-WPRE-pA | N/A |
| pAAV-hSyn-GOI | N/A |
| pLJM1-cmv-cOpn5 | N/A |
| pLJM1-cmv-tOpn5 | N/A |
| pLJM1-cmv-hOPN5 | N/A |
| pLJM1-cmv-mOpn5 | N/A |
| pLJM1-cmv-V5-Opn5 | N/A |
| pLJM1-cmv-cOpn5-T2A-eGFP | N/A |
| PAAV-hSyn-cOpn5-T2A-eGFP-WPR-pA | N/A |
| PAAV-GfaABC1D-cOpn5-T2A-eGFP-WPR-pA | N/A |
| pAAV-EF1a-DIO-cOpn5-T2A-eGFP-WPRE-pA | N/A |
| PAAV-GfaABC1D-cOpn5-T2A-mCherry-WPR-pA | N/A |

TABLE 3
Virus Strains

| | |
|---|---|
| Lenti-cmv-cOpn5-puro | Chinese Institute for |
| | Brain Research, Beijing |
| Lenti-cmv-hOPN5-puro | Chinese Institute for |
| | Brain Research, Beijing |
| Lenti-cmv-tOpn5-puro | Chinese Institute for |
| | Brain Research, Beijing |
| Lenti-cmv-mOpn5-puro | Chinese Institute for |
| | Brain Research, Beijing |
| Lenti-cmv- hM3Dq -puro | Chinese Institute for |
| | Brain Research, Beijing |
| AAV2/9-EF1a-DIO-cOpn5-T2A-eGFP | Chinese Institute for |
| | Brain Research, Beijing |
| AAV2/9-hSyn-cOpn5-T2A-eGFP | Chinese Institute for |
| | Brain Research, Beijing |
| AAV2/9-Ef1a-DIO-cOpn5-T2A-eGFP | Chinese Institute for |
| | Brain Research, Beijing |
| AAV2/8-GFaABC1D-cOpn5-T2A-eGFP | Chinese Institute for |
| | Brain Research, Beijing |
| AAV2/8-GfaABC1D-cOpn5-T2A-mCherry | Chinese Institute for |
| | Brain Research, Beijing |
| AAV2/9-EF1a-EGFP | Chinese Institute for |
| | Brain Research, Beijing |
| AAV2-EF1α-DIO-GCaMP6m | Chinese Institute for |
| | Brain Research, Beijing |
| AAV2/9-GfaABC1D-ATP1.0 | WZ Biosciences Inc. |
| | Cat. # YL006003-AV9 |
| AAV9-hSyn-NES-jRGECO1a-WPRE | WZ Biosciences Inc. |
| | Cat. # BS8-NOAAAV9 |
| AAV2/9-mCaMKIIa-jGCaMP7b-WPRE-pA | Shanghai Taitool |
| | Bioscience Co., Ltd |
| | Cat. # S0712-9-H20 |

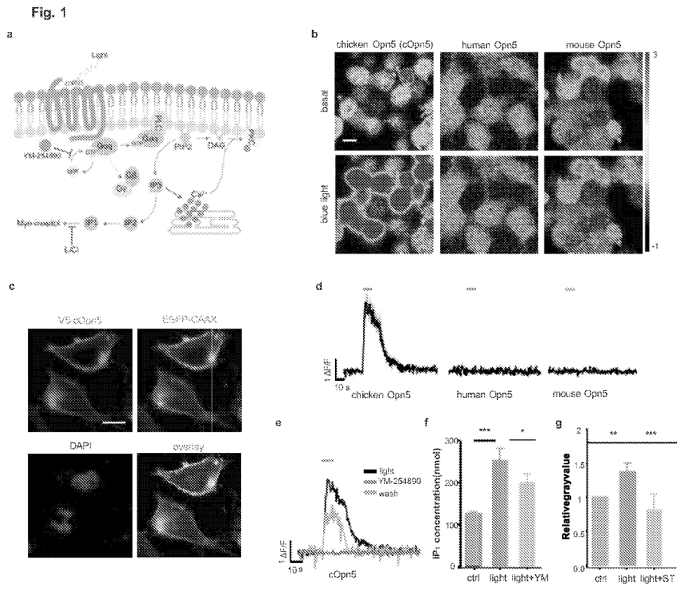

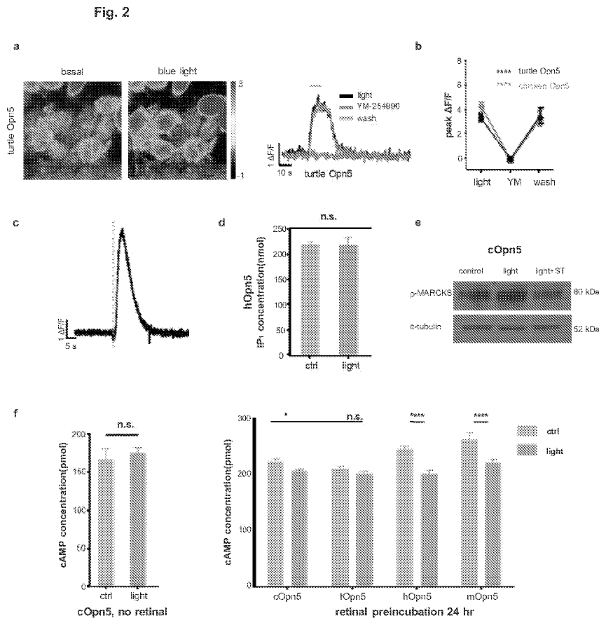

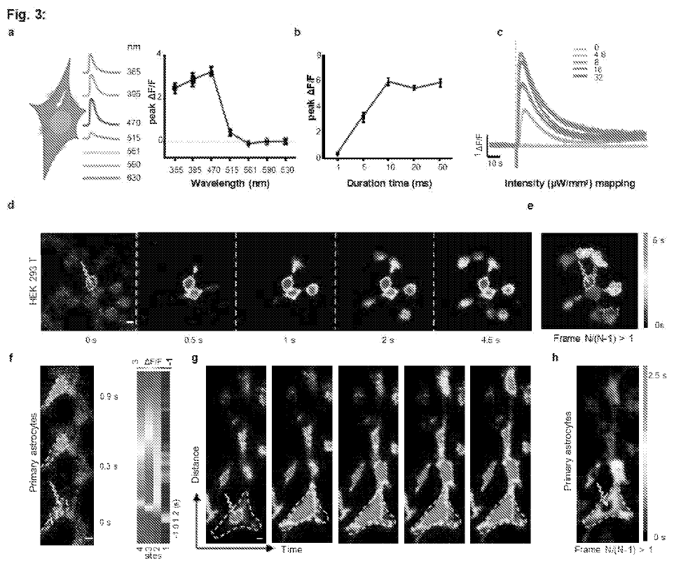

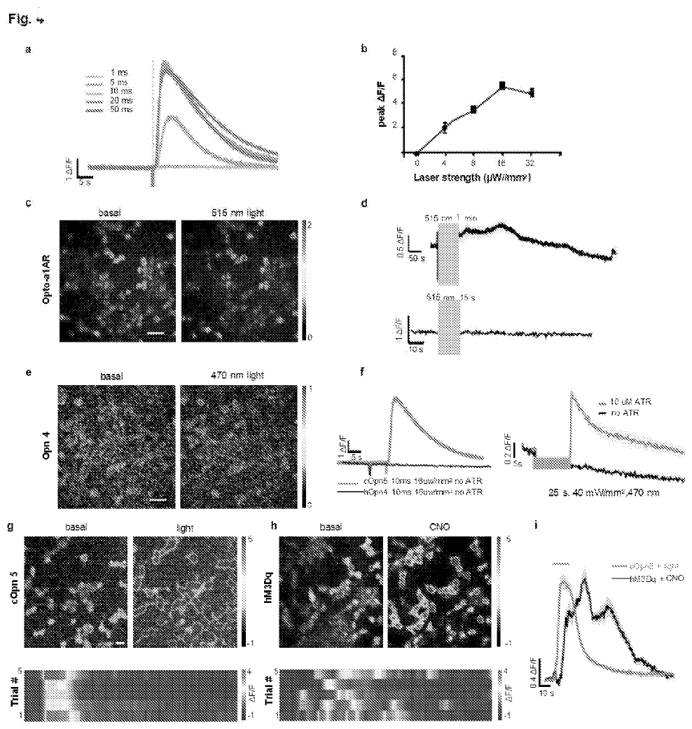

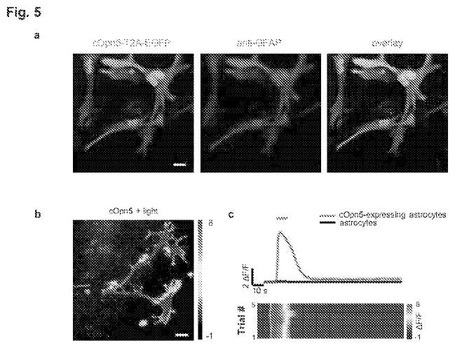

TABLE 4
Light excitation sources

| | | | |
|---|---|---|---|
| FIGS. 1f, 1g, | 470 nm | Thorlabs | M470L3 |
| FIGS. 2c, 2d, 2e, | mounted LED | | |
| 2f | | | |
| FIGS. 3b, 3c; | | | |
| FIGS. 4a, 4b, 4e, | | | |
| 4f, 4h | | | |
| FIGS. 5a, 5e, 5f, | | | |
| 5g | | | |
| FIGS. 1b, 1d, 1e; | 488 nm | Nikon | A1R MP |
| FIGS. 2a, 2b | from microscope | | |
| FIGS. 3d, 3f, 3g; | light source | | |
| FIG. 3a | 365 nm | LG3535 | wavelength |
| | mounted LED | | coverage: |
| | | | 360-370 nm |
| FIG. 3a | 395 nm | LG3535 | wavelength |
| | mounted LED | | coverage: |
| | | | 390-400 nm |
| FIG. 3a | 561 nm | Changchun New | MGL-FN-561 |
| | laser | Industries | |
| | | Optoelectronics | |
| | | Technology, | |
| | | China | |
| FIG. 3a | 590 nm | CREE XP-E2 | wavelength |
| | mounted LED | | coverage: |
| | | | 570-615 nm |
| FIG. 3a | 630 nm | CREE XP-E2 | wavelength |
| | mounted LED | | coverage: |
| | | | 615-660 nm |
| FIGS. 4c, 4d | 515 nm | Changchun New | MGL-F-515 |
| | laser | Industries | |
| | | Optoelectronics | |
| | | Technology, | |
| | | China | |

TABLE 5
Microscope equipments

| | | | |
|---|---|---|---|
| FIGS. 1b, 1d, 1e; | Multiphoton | Nikon | A1R MP |
| FIGS. 2a, 2b | confocal | | |
| | microscopes | | |
| FIGS. 3a, 3b, 3c; | Spinning Disk | Nikon | ECLIPASE Ti |
| FIG. 4a, 4b, 4c, | | | |
| 4d, 4e, 4f | | | |
| FIG. 5a | Confocal laser | Zeiss | LSM 880 |
| | scanning | | |
| | microscope | | |

TABLE 6
Statistical analysis:

| | | n per | | |
|---|---|---|---|---|
| FIG. | Conditions | group | Analysis | P value |
| 1f | ctrl vs. light | 4, 4 | Tukey's multiple | P < 0.0001 |
| | | | comparisons test | |
| | light vs. | 4, 4 | Tukey's multiple | P = 0.0128 |
| | light + | | comparisons test | |
| | YM-254890 | | | |
| 1g | ctrl vs. light | 4, 4 | Tukey's multiple | P = 0.0096 |
| | | | comparisons test | |
| | light vs. | 4, 4 | Tukey's multiple | P = 0.0004 |
| | light + | | comparisons test | |
| | staurosporine | | | |
| 2b | cOpn5 group: | 19, 15 | Tukey's multiple | P < 0.0001 |
| | light vs | | comparisons test | |
| | YM-254890 | | | |
| | cOpn5 group: | 15, 11 | Tukey's multiple | P < 0.0001 |
| | YM-254890 vs | | comparisons test | |
| | wash | | | |
| | cOpn5 group: | 19, 11 | Tukey's multiple | P = 0.2239 |
| | light vs wash | | comparisons test | |
| | tOpn5 group: | 15, 17 | Tukey's multiple | P < 0.0001 |
| | light vs | | comparisons test | |
| | YM-254890 | | | |
| | tOpn5 group: | 17, 13 | Tukey's multiple | P < 0.0001 |
| | YM-254890 vs | | comparisons test | |
| | wash | | | |
| | tOpn5 group: | 15, 13 | Tukey's multiple | P = 0.9388 |
| | light vs wash | | comparisons test | |
| 2d | ctrl vs. light | 4, 4 | Unpaired t test | P = 0.4338 |
| 2f- left | ctrl vs. light | 3, 3 | Tukey's multiple | P = 0.992 |
| | | | comparisons test | |
| 2f- Right | cOpn5 group: | 4, 4 | Tukey's multiple | P = 0.0223 |
| | ctrl vs. light | | comparisons test | |
| | tOpn5 group: | 4, 4 | Tukey's multiple | P = 0.4174 |
| | ctrl vs. light | | comparisons test | |
| | hOPN5 group: | 4, 4 | Tukey's multiple | P < 0.0001 |
| | ctrl vs. light | | comparisons test | |
| | mOpn5 group: | 4, 4 | Tukey's multiple | P < 0.0001 |
| | ctrl vs. light | | comparisons test | |

### Example 1 cOpn5 Mediates Optogenetic Activation of G_{q} Signaling

Whether heterologous expression of the Opn5 orthologs from chicken, turtles, humans and mice (which share 80-90% protein sequence identity) have the capacity to mediate blue light-induced G_{q} signaling activation within HEK 2931 cells is tested (FIG. 1a and table 7). Blue light for stimulation and the red intracellular calcium indicator Calbryte™ 630 AM dye are used to monitor the relative Ca²⁺ response (FIG. 1b). The Opn5 orthologs from chicken (cOpn5) and turtle (tOpn5) mediated an immediate and strong light-induced increase in Ca²⁺ signal (˜3 ΔF/F), whereas no light effect was observed from cells expressing the human or mouse Opn5 orthologs (FIG. 1d and FIG. 2a, b). As exemplified by the chicken ortholog, the cOpn5 co-localized with the EGFP-CAAX membrane marker, indicating that it was efficiently transported to the plasma membrane (FIG. 1c). No exogenous retinal is supplied to the culture media, which suggests that endogenous retinal is sufficient to render cOpn5 functional. The Ca²⁺ signals are resistant to the removal of extracellular Ca²⁺, thus indicating Ca²⁺ release from the intracellular stores (FIG. 2c). Preincubation of YM-254890, a highly selective G_{q} proteins inhibitor 33, reversibly abolishes the light-induced Ca²⁺ transients in both cOpn5-expressing cells (FIG. 1e). In cOpn5-, but not human OPN5-expressing cells, a light-induced increase in the level of inositol phosphate (IP1), the rapid degradation product of IP₃ is detected; moreover, the extent of this increase is reduced with the treatment of YM-254890 (FIG. 1f and FIG. 2d). In cOpn5-expressing HEK 293T cells, blue light also triggers the phosphorylation of MARCKS protein, a well-established target of PKC 34, in a PKC activity-dependent manner (FIG. 1g and FIG. 2e). By contrast, blue light illumination effectively reduces cAMP levels in cells expressing human and mouse Opn5 with retinal, but has no such effect in cells expressing cOpn5 without retinal (FIG. 2f). Collectively, these data support that blue light illumination enables the coupling of cOpn5 to the G_{q} signaling pathway in HEK 293T cells.

TABLE 7
Opsins and species

| | Alias | species | |
|---|---|---|---|
| Chicken Opn5 | cOpn5 | Gallus gallus | GenBank |
| | | | NM_001130743.1 |
| Turtle Opn5 | tOpn5 | Chelonia mydas | GenBank |
| | | | XM_007068312.4 |
| Human Opn5 | hOPN5 | Homo sapiens | GenBank |
| | | | AY377391.1 |
| Mouse Opn5 | mOpn5 | Mus musculus | GenBank |
| | | | NM_181753.4 |

FIG. 1 Shows that cOpn5 Mediates Light-Induced Strong Activation of G_{q} Signaling in HEK 293T Cells.
a, Schematic diagram of the putative intracellular signaling in response to light-induced cOpn5 activation. PLC: phospholipase C; PIP2: phosphatidylinositol-4,5-bisphosphate; IP₃: inositol-1,4,5-trisphosphate; IP₁: inositol monophosphate; DAG: diacylglycerol; PKC: protein kinase C; YM-254890: a selective G_{q} protein inhibitor.
b, Pseudocolor images of the Ca2+ signal before and after blue light stimulation (10 s; 100 μW/mm²; 488 nm) in HEK 293T cells expressing Opn5 from three species (Gallus gallus, Homo sapiens, and Mus musculus). Scale bar, 10 μm.
c, The Cy3-counterstained V5-cOpn5 fusion protein (red) was co-localized with the membrane-tagged EGFP-CAAX (green) in HEK 293T cells. DAPI counterstaining (blue) indicates cell nuclei. Scale bar, 10 μm.
d, Time courses of light-evoked Ca2+ signals for cells shown in c.
e, G_{q} protein inhibitor YM-254890 (10 nM) reversibly blocked cOpn5-mediated, light-induced Ca²⁺ signals.
f, YM suppressed the IP₁ accumulation evoked by continuous light stimulation (3 min; 100 μW/mm²; 470 nm) in cOpn5-expressing HEK 293T cells (Left). ***P<0.0001, *P=0.0128; Tukey's multiple comparisons test.
g, Phosphorylation of MARCKS in cOpn5-expressing HEK 293T cells in the control group (no light stimulation), the light stimulation group, and light+staurosporine (ST, PKC inhibitor) group. The amount of p-MARCKS in the same fraction was normalized to the amount of a-tubulin. **P=0.0096, ***P=0.0004; Tukey's multiple comparisons test.
   FIG. 2 Shows that cOpn5 Couples to G_{q} but not Gᵢ Signaling
a, Pseudocolor images of the Ca2+ signal before and after blue light stimulation (10 s; 100 μW/mm2; 488 nm) in HEK 293T cells expressing Opn5 from turtle species (Chelonia mydas). Scale bar, 10 μm (left); Time courses of light-evoked Ca2+ signals for responed cells (right)
b, Group data of the Gq protein inhibitor YM-254890 (10 nM) reversibly blocked cOpn5- and turtle Opn5-mediated, light-induced Ca2+ signals. ****P<0.0001, one way ANOVA. Error bars indicate S.E.M.
c, Time course of Ca2+ signal with photostimulation (10 ms; 16 μW/mm2; 470 nm) without extracellular Ca2+.
d, IP1 accumulation in human Opn5-expressing HEK 293T cells with or without light stimulation (Right). n.s., no significant difference; unpaired t test.
e, One representative of phosphorylation of MARCKS in cOpn5-expressing HEK 293T cells in the control group (no light stimulation), the light stimulation group, and light+staurosporine group. The amount of p-MARCKS in the same fraction was normalized to the amount of a-tubulin.
f, Light has no effect on cAMP levels (10 μM forskolin preincubation) in cOpn5-expressing HEK 293T cells without additional retinal in the medium (left panel). Right panel shows the effects of photostimulation on cAMP concentrations for HEK 293T cells expressing Opn5s from four different species following 10 μM retinal preincubation.

Error bars in d and f indicate S.E.M.

### Example 2 cOpn5-Mediated Optogenetics is Sensitive and Precise

Characterizing the light-activating properties of cOpn5 heterologously expressed in HEK 293T cells is performed. Although Opn5 is previously considered as an ultraviolet (UV)-sensitive photoreceptor²⁷, mapping with a set of wavelengths ranging 365-630 nm at a fixed light intensity of (100 μW/mm²) revealed that the 470 nm blue light elicited the strongest Ca2+ transients, with the UVA light (365 and 395 nm) being less effective and longer-wavelength visible light (561 nm or above) completely ineffective (FIG. 3a). The effects of different light durations on cOpn5-expressing HEK 293T cells are tested. Stimulating with brief light pulses (1, 5, 10, 20, 50 ms; 16 μW/mm²; 470 nm) shows that the Ca2+ response achieves the saturation mode with light duration over 10 ms (FIG. 3b). Longer light durations do not further increase the Ca2+ signal amplitude at this light intensity (16 μW/mm²; 470 nm) (FIG. 4a). Delivering 470 nm light at different intensities shows that blue light of ˜4.8 μW/mm² and 16 μW/mm² produce about half maximum and full maximum responses, respectively (FIG. 3c and FIG. 4b). Therefore, the light sensitivity of cOpn5 is 3-4 orders of magnitude higher than the reported values of the light-sensitive Gq-coupled GPCRs and even 2-3 orders higher than those of the commonly used optogenetic tool Channelrhodopsin-2 (ChR2)(Lin, 2011; Zhang et al., 2006) (table 8). Together, these results indicate that cOpn5 could function as a single-component optogenetic tool without additional retinal, and that cOpn5 is super-sensitive to blue light for its full activation requiring low light intensity (16 μW/mm²) and short duration (10 ms).

TABLE 8
Comparison cOpn5 with other optogenetic tools

| | | | | | | Need for | | |
|---|---|---|---|---|---|---|---|---|
| | Wavelength | | | Stimulation | | exogeneous | Response | |
| | λₘₐₓ (nm) | Light Sensitivity | | duration | | chemicals(retinal) | amplitude | model |
| Wild-type | 470 nm | 8-12 | mW/mm² | 2.3 ± 1.1 | ms | No | steady state: | Hippocampal |
| ChR2 ^{1, 2} | | | | | | | peak current | cell culture |
| | | | | | | | ratio: 0.4 ± | |
| | | | | | | | 0.04; (731 ± | |
| | | | | | | | 100 pA | |
| ChR2 H134R ³ | 450 nm | ~10 mW/mm² | | 0.96 ± 0.12 | ms | No | 4.47 nA | HEK 293T |
| | | (470 nm) | | | | | | |
| ChETA ⁴ | 490 nm | ~10 | mW/mm² | 0.9 ± 0.1 | ms | No | steady state: | Hippocampal |
| | | | | | | | peak current | cell culture |
| | | | | | | | ratio: 0.6 ± | |
| | | | | | | | 0.04; (645 ± | |
| | | | | | | | 47 pA | |
| ChrimsonR ⁵ | 590 nm | 4.6 | mW/mm² | 0.9 ± 0.1 | ms | No | ~300 pA | cultured |
| | | | | | | | | neurons |
| mouse | 480 nm | 1015 photons s⁻¹ | | >60 | s | 11-cis- | ~0.1 (ΔF/F) | HEK293- |
| melanopsin | | cm⁻² (500 nm) | | | | retinaldehyde | Ca²⁺ | TRPC3 cells |
| (Opn4) ⁶ | | | | | | | response | |
| | | | | | | | ampitude | |
| mouse | 488 nm | a white | | 60 | s | 11-cis retinal | ~0.25 | CHO cells |
| melanopsin | | fluorescent light | | | | | (ΔF/F) Ca²⁺ | |
| (Opn4) and | | source (intensity | | | | | response | |
| its mutants ⁷ | | undefined) | | | | | amplitude, | |
| | | | | | | | the best | |
| | | | | | | | mutant | |
| | | | | | | | Opn4^{9A} | |
| hOpn4- | 473 nm | 7 | mW/mm² | 20 | s | Unknown | ~2 (ΔF/F) | in vivo |
| human | | | | | | | Ca²⁺ event | astrocytes |
| melanopsin ⁸ | | | | | | | frequence, | |
| | | | | | | | but no | |
| | | | | | | | significant | |
| | | | | | | | change in | |
| | | | | | | | Ca²⁺ | |
| | | | | | | | amplitude | |
| opto-α1AR ⁹ | 500 nm | 7 | mW/mm² | 60 | s | No | ~0.227 | HEK cells |
| | | | | | | | (ΔF/F) Ca²⁺ | |
| | | | | | | | response | |
| | | | | | | | ampitude | |
| opto-α1AR ¹⁰ | 473 nm | 20 Hz, 45-ms | | 5 | min | No | >20% | in vitro |
| | | light pulses, | | | | | increase in | astrocytes |
| | | 5 mW | | | | | sIPSC | |
| | | | | | | | frequency | |
| human | 470 nm | 40 | mW/mm² | 25 | s | ATR | ~0.646 | HEK 293T |
| melanopsin | | | | | | | (ΔF/F) Ca²⁺ | |
| | | | | | | | response | |
| | | | | | | | ampitude | |
| opto-α1AR | 510 nm | 7 | mW/mm² | 60 | s | No | ~0.5 (ΔF/F) | HEK 293T |
| | | | | | | | Ca²⁺ | |
| | | | | | | | response | |
| | | | | | | | ampitude | |
| hM3Dq | | | | | | CNO | ~1.6 (ΔF/F) | HEK 293T |
| | | | | | | | Ca²⁺ event | |
| | | | | | | | frequence, | |
| | | | | | | | but no | |
| | | | | | | | significant | |
| | | | | | | | change in | |
| | | | | | | | Ca²⁺ | |
| | | | | | | | amplitude | |
| cOpn5 | 470 nm | 16 | μW/mm² | 10 | ms | No | ~3.0 (ΔF/F) | HEK 293T |
| | | | | | | | Ca²⁺ | cells |
| | | | | | | | response | |
| | | | | | | | amplitude | |
| | 470 nm | 0.026 | μW/mm² | >2 | s | No | ~1 (ΔE/F) | HEK 293T |
| | | | | | | | Ca²⁺ | cells |
| | | | | | | | response | |
| | | | | | | | amplitude | |

The performance of cOpn5 to that of opto-a1AR, a chimera GPCR engineered by mixing rhodopsin with G_{q}-coupled adrenergic receptor is compared. Following the protocol in a previous report¹⁴, it is found that very long exposure of strong illumination (60 s; 7 mW/mm²) is required to trigger a slow and small (˜0.5 ΔF/F) Ca²⁺ signal increase in opto-a1AR-expressing HEK 2931 cells, and 15 s illumination is inefficient (FIG. 4c, d). The performance of cOpn5 to that of opn4, a natural opsin which was reported as a tool for G_{q} signaling activating is also compared. It is found that long exposure of strong illumination (25 s; 40 mW/mm²) and additional retinal are required to trigger a slow (˜1 ΔF/F) Ca²⁺ signal increase in opn4-expressing HEK 2931 cells (FIG. 4e, f). Therefore, compared with existing opsin-based tools (opto-a1AR and opn4), cOpn5 is much more light-sensitive (˜3 orders more sensitive), requires much shorter time exposure (10 ms vs. 60 s), and produces stronger responses.

The performance of cOpn5 to that of the popular G_{q}-coupled chemogenetic tool hM3Dq, which is activated by adding the exogenous small molecule ligand clozapine-N-oxide (CNO)³⁷⁻³⁹ is compared. Light-induced activation of cOpn5-expressing HEK 293T cells has a similar peak response amplitude of the Ca²⁺ signal as CNO-induced activation of hM3Dq-expressing HEK 293T cells. Meanwhile, cOpn5-expressing HEK 293T cells have faster and temporally more precise response, as well as more rapid recovery time than hM3Dq-expressing HEK 293T cells (FIG. 4g-i). These results indicate that cOpn5-mediated optogenetics are more controllable in temporal accuracy than those of hM3Dq.

cOpn5 optogenetics allows spatially precise control of cellular activity. Restricting brief light stimulation (63 ms) into a subcellular region of individual cOpn5-expressing HEK 293T cell results in the immediate activation of single cell. Interestingly, in high cell confluence area, the Ca²⁺ signals propagated to surrounding cells, thus suggesting intercellular communication among HEK 293T cells through a yet-to-identified mechanism (FIG. 3d, e). The findings are extended into primary cell cultures. cOpn5 is expressed in primary astrocyte cultures prepared from the neonatal mouse brain with AAV vectors for bicistronic expression of cOpn5 and the EGFP marker protein (FIG. 5a). Using the Calbryte 630 AM dye to monitor Ca²⁺ levels, it is found that blue light illumination of cOpn5-expressing astrocytes produces strong Ca²⁺ transients (˜8 ΔF/F) (FIG. 5b, c). If the light stimulation (63 ms) is precisely restricted to only subcellular region of an individual cOpn5-expressing astrocyte, Ca²⁺ signal propagation within the individual cell is observed (FIG. 3f). Resembling the tests in HEK 293T cells, wave-like propagation of Ca²⁺ signals from the stimulated astrocyte that proceeded gradually to more distal, non-stimulated, astrocytes is observed (FIG. 3g, h). These experiments thus demonstrate that cOpn5 optogenetics allows precise spatial control, and suggest that it may be useful to study the dynamics of astrocytic networks, which is initially discovered using neurochemical and mechanical stimulation^{40,41}.

FIG. 3 Shows that cOpn5 Sensitively Mediates Optical Control of G_{q} Signaling with High Temporal and Spatial Resolution.
a, Schematic diagram of selected wavelengths (365, 395, 470, 515, 561, 590, and 630 nm; left panel) and the amplitudes of Ca²⁺ signal of cOpn5-expressing HEK 293T cells in response to light stimulation with different wavelengths (2 s; 100 μW/mm²; right panel). Error bars indicate S.E.M.
b, The response magnitude under different duration of light stimulation (1, 5, 10, 20, or 50 ms; 16 μW/mm²; 470 nm). Error bars indicate S.E.M.
c, Time course of cOpn5-mediated Ca²⁺ signals under different light intensity (0, 4.8, 8, 16, or 32 μW/mm²; 10 ms; 470 nm; for 10 ms 16 μW/mm² stimulation, 10% peak activation=1.36±0.55 s; 90% peak activation=2.37±0.87 s; decay time τ=18.66±4.98 s, mean±S.E.M.; n=10 cells).
d, Images of light-induced (63 ms; 17 μW; arrow points to the stimulation region) Ca²⁺ signal propagation in cOpn5-expressing HEK 293T cells. Scale bar, 10 μm.
e, Pseudocolor images showing the process of Ca²⁺ signal propagation across time of d (frame N/(N−1)>1). Frame interval was 500 ms and each frame is counted once.
f, Images of light-induced Ca²⁺ signal propagation in a single cOpn5-expressing primary astrocyte stimulated in a subcellular region (stimulation size 4×4 μm² and frame interval 300 ms). Scale bar, 10 μm.
g, Images of light-induced Ca²⁺ signal propagation in cOpn5-expressing primary astrocytes. Scale bar, 10 μm.
h, Pseudocolor images showing process of Ca²⁺ signal propagation across time of g (frame N/(N−1)>1). Frame interval was 500 ms and each frame is counted once.
   FIG. 4 Shows that cOpn5 Mediates More Rapid and Sensitive Response to Light than Opto-a1AR, hM3Dq or Opn4.
a, Time course of Ca²⁺ signal with light pulses (16 μW/mm²; 470 nm; 1, 5, 10, 20, or 50 ms).
b, The response magnitude under different light intensities (0, 4.8, 8, 16, or 32 μW/mm²) at 10 ms, 470 nm.
c, Pseudocolor images of the baseline and peak Ca²⁺ signals (ΔF/F0) in opto-a1AR-expressing HEK 293T cells. The medium buffer contains 10 μM all-trans-retinal. Scale bar, 30 μm.
d, Effect of 60 s light stimulation on the Ca²⁺ in opto-a1AR-expressing HEK 293T cells (n=15 cells; upper panel) and the lack of effect by 15 s light stimulation on Ca²⁺ signals (lower panel).
e, Pseudocolor images of the baseline and peak Ca²⁺ signals (ΔF/F0) in human OPN4-expressing HEK 293T cells. The medium buffer contains 10 μM all-trans-retinal. Scale bar, 30 μm.
f, Effect of 25 s light stimulation on the Ca²⁺ in OPN4-expressing HEK 293T cells within 10 μM ATR (n=12 cells; red line) and the lack of effect by without ATR on Ca²⁺ signals (black panel).
g, Effects of light stimulation on the Ca²⁺ signals in cOpn5-expressing HEK 293T cells. Upper panels show pseudocolor images of baseline and peak response. Lower panel shows the heat map of Ca²⁺ signals evoked by cOpn5-mediated optogenetic stimulation in HEK 293T cells expressing cOpn5 across 5 consecutive trials. Scale bar, 20 μm.
h, Effect of chemogenetic stimulation on the Ca²⁺ signals in hM3Dq-expressing HEK 293T cells.
i, Time courses of Ca²⁺ signals evoked by cOpn5-mediated optogenetic stimulation (10 s) and hM3Dq-mediated chemogenetic stimulation using CNO puff (100 nM; 10 s), respectively.
   FIG. 5 Shows that cOpn5 Effectively Mediates the Activation of Astrocytes.
a, cOpn5 was expressed in cultured primary astrocytes using AAV-cOpn5-T2A-EGFP (green). Astrocyte identity was confirmed by GFAP immunostaining (red). Scale bar, 20 μm.
b, Pseudocolor images of the baseline and peak Ca²⁺ signals following light stimulation of cOpn5-expressing astrocytes. Scale bar, 20 μm.
c, Plot of Ca²⁺ signals and heat map representation of Ca²⁺ signals across trials (n=25 cells).

### Example 3 Optogenetic Visual Restoration Using Light-Sensitive Gq-Coupled Neuropsin (Opsin 5)

### Animal Model:

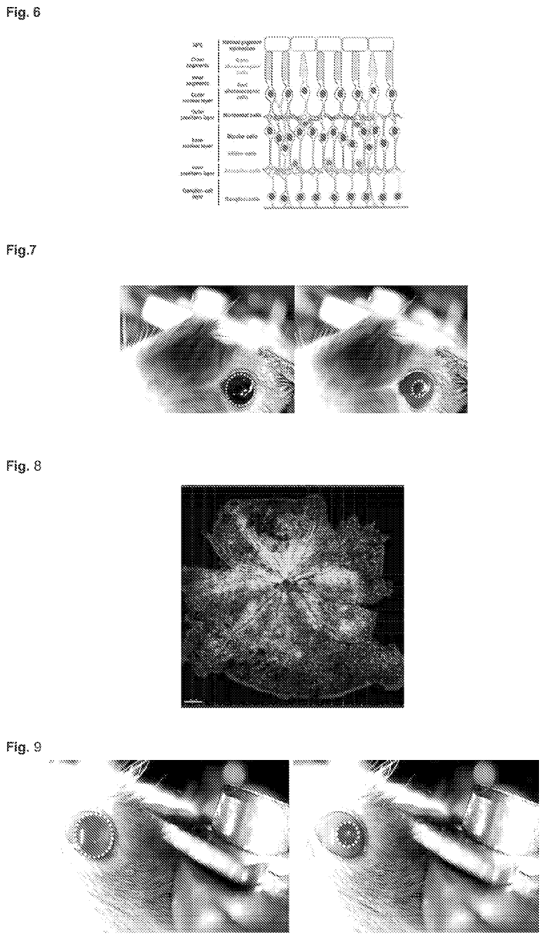

1. Health retina contains several cell layers: retinal pigment epithelium, cone photoreceptor cells, rod photoreceptor cells, horizontal cells, bipolar cells, Müller cells, Amacrine cells, Ganglion cells (FIG. 6). Methylnitrosourea (MNU) results photoreceptor (rod and cone photoreceptors) damage and then induces retinal degeneration in animals. We use MNU induce mice retinal degeneration as an animal model. Retinal degeneration induced by a single intraperitoneal injection of MNU with the dose of 60 mg/kg body weight.
2. C3H/HeNCrl Mice are genetic retinal degeneration models. This strain has a characteristic that homozygous for Pde6b^{rd1} mutation causing retinal degeneration.

We use the pupillary light response with head fixed mice to test whether the animal could sense the light, and we use AAV vectors expressing cOpn5 in mice retinal ganglion cells to rescue these two mice models. The mice recover pupillary light response demonstrates our cOpn5-mediated approach of blindness treatment.

### Experiments and Results

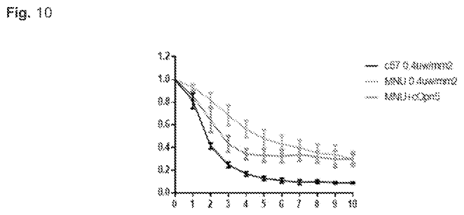

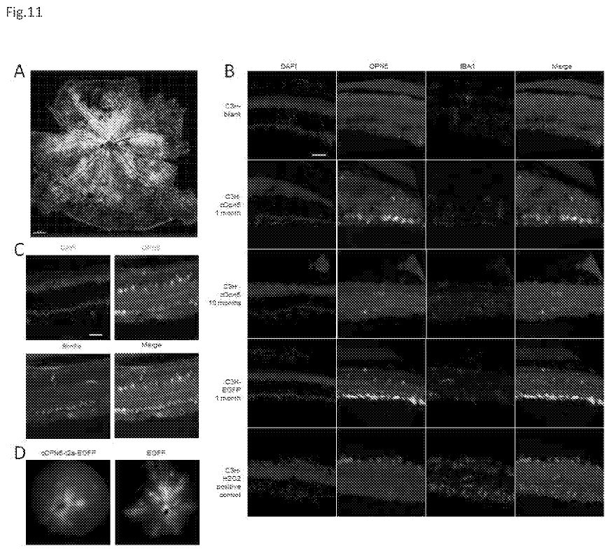

1. We use camera with IR blocking to automatically acquire images of head fixed mice pupils. Adjust optical fiber to make sure the light (470 nm LED light source) shoots straight on mice pupils with the same light intensity.
2. Normal mice before MNU-treated have rapid pupillary light response (FIG. 7). C3H/HeNCrl inbred Mice didn't have pupillary light response (FIG. 7).
3. C3H/HeNCrl or MNU treated retinal degeneration mice lost functions of pupillary light response
4. We use AAV vector expressed cOpn5-t2a-EGFP in mice retinal ganglion cells, the image shows EGFP in the whole retina after 4 weeks after AAV injection (FIG. 8).
5. After cOpn5 expressed in the mice retinal ganglion cells, we do the pupillary light response test again. The MNU mice-treated recovered the pupillary light response (FIG. 9). The C3H/HeNCrl mice gain the ability of pupillary light response (FIG. 9).
6. FIG. 10 shows in pupillary light response test: normal mice (black solid line) pupil size rapid decrease in response to light (X-axis: time (second); Y-axis: normalized pupil size). After MNU treatment, the mice lost functions in pupillary light response test (gray solid line). When using AAV vectors expressing cOpn5 in the retinal ganglion cells (RGC) of these MNU treated mice 4 weeks later, the mice partially recovered the pupillary light response capability (middle solid line).

These results demonstrate our approach that expressing cOpn5 in animal retinal ganglion cells can recover retinal degeneration.

### Example 4

Experiments description: the following table 9 is a partial list of cOpn5 orthologs from vertebrata tested in the present invention. Whole genes of all reported opsin5 orthologs from vertebrata (the vertebrates subphylum, including rotundia, cartilaginous fishes, bony fishes, Amphibia, reptila, ornitha and mammals) are synthetized, and expressed in HEK 293T cells. Calcium imaging with or without 470 nm blue light stimulation is performed to test the sensitivity of the opsin 5 orthologs in response to light. The time course of light-induced calcium signal reveal the activated degree of G_{q} signaling pathway and the sensitivity of these orthologs.

TABLE 9

| Entry | Entry name | Activity | Protein names | Gene names |
|---|---|---|---|---|
| E0R7P4 | E0R7P4_XENLA | | Opn5 (Opsin) | opn5.L opn5 |
| | | | | XELAEV_18028134 mg |
| A0A455SGG5 | A0A455SGG5_9EUPU | | Opsin-5A | opn5a |
| A0A4Z2FX25 | A0A4Z2FX25_9TELE | | Opsin-5 | OPN5_5 EYF80_044932 |
| A0A4Z2FH27 | A0A4Z2FH27_9TELE | | Opsin-5 | OPN5_4 EYF80_049299 |
| A0A4Z2IDU8 | A0A4Z2IDU8_9TELE | | Opsin-5 | OPN5_3 EYF80_013671 |
| A0A4Z2H0H0 | A0A4Z2H0H0_9TELE | | Opsin-5 | OPN5_2 EYF80_030918 |
| A0A218USZ0 | A0A218USZ0_9PASE | | Opsin-5 | OPN5_1 RLOC_00008660 |
| A0A4Z2FVH4 | A0A4Z2FVH4_9TELE | | Opsin-5 | Opn5_0 EYF80_044930 |
| A0A4Z2HA58 | A0A4Z2HA58_9TELE | | Opsin-5 | OPN5_0 EYF80_027087 |
| A0A218UGP1 | A0A218UGP1_9PASE | | Opsin-5 | OPN5_0 RLOC_00005796 |
| G1L3V2 | G1L3V2_AILME | | Opsin 5 | OPN5 |
| A0A6P4X9I3 | A0A6P4X9I3_PANPR | | opsin-5 | OPN5 |
| A0A1S2ZDX4 | A0A1S2ZDX4_ERIEU | | opsin-5 | OPN5 |
| A0A2I4C032 | A0A2I4C032_9TELE | | opsin-5 | opn5 |
| U3JFW4 | U3JFW4_FICAL | | Opsin 5 | OPN5 |
| A0A2Y9NPU7 | A0A2Y9NPU7_DELLE | | opsin-5 | OPN5 |
| A0A1U7U6G6 | A0A1U7U6G6_CARSF | | opsin-5 | OPN5 |
| A0A6I9I544 | A0A6I9I544_VICPA | | opsin-5 | OPN5 |
| M3YLS7 | M3YLS7_MUSPF | | G_PROTEIN_RECEP_F1_2 | OPN5 |
| | | | domain-containing protein | |
| A0A5F9CCV1 | A0A5F9CCV1_RABIT | | Opsin 5 | OPN5 |
| A0A671EF51 | A0A671EF51_RHIFE | | Opsin 5 | OPN5 |
| A0A6P6I1D4 | A0A6P6I1D4_PUMCO | | opsin-5 | OPN5 |
| G3RKG7 | G3RKG7_GORGO | | Opsin 5 | OPN5 |
| G1NYV5 | G1NYV5_MYOLU | | Opsin 5 | OPN5 |
| A0A6J2L9P4 | A0A6J2L9P4_9CHIR | | opsin-5 | OPN5 |
| A0A2K6AXI4 | A0A2K6AXI4_MACNE | | Opsin 5 | OPN5 |
| A0A6J3JM90 | A0A6J3JM90_SAPAP | | opsin-5 | OPN5 |
| A0A452TE17 | A0A452TE17_URSMA | | Opsin 5 | OPN5 |
| A0A384C5D1 | A0A384C5D1_URSMA | | opsin-5 | OPN5 |
| A0A2K5U4B7 | A0A2K5U4B7_MACFA | | Opsin 5 | OPN5 |
| A0A2I3MZV4 | A0A2I3MZV4_PAPAN | | Opsin-5 | OPN5 |
| A0A2K5U4B3 | A0A2K5U4B3_MACFA | | Opsin 5 | OPN5 |
| Q6U736 | OPN5_HUMAN | reviewed | Opsin-5 (G-protein coupled | OPN5 GPR136 PGR12 |
| | | | receptor 136) (G-protein | TMEM13 |
| | | | coupled receptor PGR12) | |
| | | | (Neuropsin) (Transmembrane | |
| | | | protein 13) | |
| F6UZB2 | F6UZB2_XENTR | | Opsin 5 | opn5 |
| A0A4W3IAF8 | A0A4W3IAF8_CALMI | | G_PROTEIN_RECEP_F1_2 | opn5 |
| | | | domain-containing protein | |
| A0A6P7HHM6 | A0A6P7HHM6_9TELE | | opsin-5 | opn5 |
| H3B1A3 | H3B1A3_LATCH | | G_PROTEIN_RECEP_F1_2 | OPN5 |
| | | | domain-containing protein | |
| A0A4W3I3H5 | A0A4W3I3H5_CALMI | | G_PROTEIN_RECEP_F1_2 | opn5 |
| | | | domain-containing protein | |
| A0A1S3MCD5 | A0A1S3MCD5_SALSA | | opsin-5 | opn5 |
| A0A4W4FPG5 | A0A4W4FPG5_ELEEL | | G_PROTEIN_RECEP_F1_2 | opn5 |
| | | | domain-containing protein | |
| A0A6P7LVJ1 | A0A6P7LVJ1_BETSP | | opsin-5 isoform X2 | opn5 |
| A0A6P7LVE3 | A0A6P7LVE3_BETSP | | opsin-5 isoform X1 | opn5 |
| A0A674IKC9 | A0A674IKC9_TERCA | | Opsin 5 | OPN5 |
| A0A674IMF3 | A0A674IMF3_TERCA | | Opsin 5 | OPN5 |
| F1NEY2 | F1NEY2_CHICK | | G_PROTEIN_RECEP_F1_2 | OPN5 |
| | | | domain-containing protein | |
| E6P6L8 | E6P6L8_DANRE | | Opsin 5 | opn5 |
| A0A671TVX9 | A0A671TVX9_SPAAU | | Opsin 5 | opn5 |
| A0A7M4FP40 | A0A7M4FP40_CROPO | | Opsin 5 | OPN5 |
| A0A671TVX4 | A0A671TVX4_SPAAU | | Opsin 5 | opn5 |
| A0A6I9Y3G3 | A0A6I9Y3G3_9SAUR | | opsin-5 | OPN5 |
| G1KNV3 | G1KNV3_ANOCA | | G_PROTEIN_RECEP_F1_2 | OPN5 |
| | | | domain-containing protein | |
| A0A493T549 | A0A493T549_ANAPP | | Opsin 5 | OPN5 |
| A0A6I9HELA | A0A6I9HELA_GEOFO | | opsin-5 isoform X2 | OPN5 |
| A0A218UPZ6 | A0A218UPZ6_9PASE | | Opsin-5 | OPN5 RLOC_00008263 |
| D8KW68 | D8KW68_ZONAL | | Opsin 5 | OPN5 |
| A0A663EIX5 | A0A663EIX5_AQUCH | | Opsin 5 | OPN5 |
| G1NNA7 | G1NNA7_MELGA | | Opsin 5 | OPN5 |
| A0A663EK31 | A0A663EK31_AQUCH | | Opsin 5 | OPN5 |
| A0A6J0Z1K0 | A0A6J0Z1K0_ODOVR | | opsin-5 | OPN5 |
| A0A6P3J431 | A0A6P3J431_BISBI | | opsin-5 | OPN5 |
| A0A2K5R3Y3 | A0A2K5R3Y3_CEBIM | | Opsin 5 | OPN5 |
| A0A671EF86 | A0A671EF86_RHIFE | | Opsin 5 | OPN5 mRhiFer1_012304 |
| A0A6I9ZSG3 | A0A6I9ZSG3_ACIJB | | opsin-5 | OPN5 |
| A0A2K6R8Q2 | A0A2K6R8Q2_RHIRO | | G_PROTEIN_RECEP_F1_2 | OPN5 |
| | | | domain-containing protein | |
| A0A4W2GZA3 | A0A4W2GZA3_BOBOX | | Opsin 5 | OPN5 |
| U3J4Q3 | U3J4Q3_ANAPP | | Opsin 5 | OPN5 |
| A0A6J2V8J5 | A0A6J2V8J5_CHACN | | opsin-5 | opn5 |
| A0A493T6P1 | A0A493T6P1_ANAPP | | Opsin 5 | OPN5 |
| A0A6J2J0L1 | A0A6J2J0L1_9PASS | | opsin-5 | OPN5 |
| A0A6P9CE92 | A0A6P9CE92_PANGU | | opsin-5 | OPN5 |
| A0A6J1V4P8 | A0A6J1V4P8_9SAUR | | opsin-5 | OPN5 |
| A0A288HLV3 | A0A288HLV3_ANSCY | | Opsin-5 | OPN5 |
| A0A151PID4 | A0A151PID4_ALLMI | | Opsin-5 | OPN5 Y1Q_0020212 |
| Q5RIV6 | Q5RIV6_DANRE | | Opsin 5 (Teleost neuropsin) | opn5 |
| D6RDV4 | D6RDV4_HUMAN | | Opsin-5 | OPN5 |
| J3KPQ2 | J3KPQ2_HUMAN | | Opsin-5 | OPN5 hCG_1642475 |
| F6XNY7 | F6XNY7_ORNAN | | Opsin 5 | OPN5 |
| A0A2K6FXK2 | A0A2K6FXK2_PROCO | | Opsin 5 | OPN5 |
| E2RPZ0 | E2RPZ0_CANLF | | Opsin 5 | OPN5 |
| A0A2K6V732 | A0A2K6V732_SAIBB | | Opsin 5 | OPN5 |
| A0A4X2K722 | A0A4X2K722_VOMUR | | Opsin 5 | OPN5 |
| A0A6P5KYE6 | A0A6P5KYE6_PHACI | | opsin-5 | OPN5 |
| A0A2K6FXJ4 | A0A2K6FXJ4_PROCO | | Opsin 5 | OPN5 |
| A0A4X2JZA4 | A0A4X2JZA4_VOMUR | | Opsin 5 | OPN5 |
| G1SX53 | G1SX53_RABIT | | Opsin 5 | OPN5 |
| A0A2U3WI94 | A0A2U3WI94_ODORO | | opsin-5 | OPN5 |
| A0A2K6V724 | A0A2K6V724_SAIBB | | Opsin 5 | OPN5 |
| A0A3Q7XKC9 | A0A3Q7XKC9_URSAR | | opsin-5 | OPN5 |
| A0A452RBH1 | A0A452RBH1_URSAM | | Opsin 5 | OPN5 |
| G1QVY1 | G1QVY1_NOMLE | | G_PROTEIN_RECEP_F1_2 | OPN5 |
| | | | domain-containing protein | |
| G1QVX6 | G1QVX6_NOMLE | | G_PROTEIN_RECEP_F1_2 | OPN5 |
| | | | domain-containing protein | |
| G3SJY5 | G3SJY5_GORGO | | Opsin 5 | OPN5 |
| A0A7N9CSX2 | A0A7N9CSX2_MACFA | | Opsin 5 | OPN5 |
| A0A384B2Q9 | A0A384B2Q9_BALAS | | opsin-5 | OPN5 |
| A0A2K6L978 | A0A2K6L978_RHIBE | | G_PROTEIN_RECEP_F1_2 | OPN5 |
| | | | domain-containing protein | |
| A0A2K6AXE7 | A0A2K6AXE7_MACNE | | Opsin 5 | OPN5 |
| A0A2J8P0S9 | A0A2J8P0S9_PANTR | | Opsin 5 | OPN5 |
| W5PR22 | W5PR22_SHEEP | | G_PROTEIN_RECEP_F1_2 | OPN5 |
| | | | domain-containing protein | |
| F7DJ88 | F7DJ88_CALJA | | G_PROTEIN_RECEP_F1_2 | OPN5 |
| | | | domain-containing protein | |
| A0A2K5R3Z8 | A0A2K5R3Z8_CEBIM | | Opsin 5 | OPN5 |
| F6PHB6 | F6PHB6_CALJA | | G_PROTEIN_RECEP_F1_2 | OPN5 |
| | | | domain-containing protein | |
| M3WMC9 | M3WMC9_FELCA | | Opsin 5 | OPN5 |
| A0A2K5L5D5 | A0A2K5L5D5_CERAT | | Opsin 5 | OPN5 |
| E1BNN4 | E1BNN4_BOVIN | | Opsin 5 | OPN5 |
| F6RFW7 | F6RFW7_MACMU | | Opsin 5 | OPN5 |
| A0A2J8RKP9 | A0A2J8RKP9_PONAB | | Uncharacterized protein | OPN5 |
| A0A3Q7RXX8 | A0A3Q7RXX8_VULVU | | opsin-5 | OPN5 |
| A0A2K5L5D9 | A0A2K5L5D9_CERAT | | Opsin 5 | OPN5 |
| H0WJY2 | H0WJY2_OTOGA | | Opsin 5 | OPN5 |
| A0A6P3ENQ6 | A0A6P3ENQ6_SHEEP | | opsin-5 | OPN5 |
| G3UA68 | G3UA68_LOXAF | | G_PROTEIN_RECEP_F1_2 | OPN5 |
| | | | domain-containing protein | |
| A0A6P5DVT1 | A0A6P5DVT1_BOSIN | | opsin-5 | OPN5 |
| A0A0D9RJS4 | A0A0D9RJS4_CHLSB | | Opsin 5 | OPN5 |
| I3LTK7 | I3LTK7_PIG | | Opsin 5 | OPN5 |
| A0A2K5Z564 | A0A2K5Z564_MANLE | | Opsin 5 | OPN5 |
| A0A5G2R7I1 | A0A5G2R7I1_PIG | | Opsin 5 | OPN5 |
| A0A6I9JGH7 | A0A6I9JGH7_CHRAS | | opsin-5 | OPN5 |
| A0A2K5Z517 | A0A2K5Z517_MANLE | | Opsin 5 | OPN5 |
| A0A452FM79 | A0A452FM79_CAPHI | | Opsin 5 | OPN5 |
| F6SJH5 | F6SJH5_HORSE | | Opsin 5 | OPN5 |
| A0A2R9BTW5 | A0A2R9BTW5_PANPA | | Opsin 5 | OPN5 |
| A0A2Y9FNI2 | A0A2Y9FNI2_PHYMC | | opsin-5 | OPN5 |
| A0A340WR35 | A0A340WR35_LIPVE | | opsin-5 | OPN5 |
| A0A6J2DJL3 | A0A6J2DJL3_ZALCA | | opsin-5 | OPN5 |
| A0A4X1UZM3 | A0A4X1UZM3_PIG | | G_PROTEIN_RECEP_F1_2 | OPN5 |
| | | | domain-containing protein | |
| A0A673TX31 | A0A673TX31_SURSU | | Opsin 5 | OPN5 |
| A0A341D5X7 | A0A341D5X7_NEOAA | | opsin-5 | OPN5 |
| A0A667FWA1 | A0A667FWA1_LYNCA | | Opsin 5 | OPN5 |
| A0A5B7H9S7 | A0A5B7H9S7_PORTR | | Opsin-5 | Opn5 E2C01_063173 |
| A0A337SC50 | A0A337SC50_FELCA | | Opsin 5 | OPN5 |
| H2RD19 | H2RD19_PANTR | | Opsin 5 | OPN5 |
| A0A2U3X849 | A0A2U3X849_LEPWE | | opsin-5 | OPN5 |
| G3THK6 | G3THK6_LOXAF | | G_PROTEIN_RECEP_F1_2 | OPN5 |
| | | | domain-containing protein | |
| A0A2U3V1E1 | A0A2U3V1E1_TURTR | | opsin-5 | OPN5 |
| A0A096NIY4 | A0A096NIY4_PAPAN | | Opsin-5 | OPN5 |
| A0A6P3PSZ2 | A0A6P3PSZ2_PTEVA | | opsin-5 | OPN5 |
| A0A2K5EFR2 | A0A2K5EFR2_AOTNA | | Opsin 5 | OPN5 |
| A0A3Q7QKC2 | A0A3Q7QKC2_CALUR | | opsin-5 | OPN5 |
| F7DVJ0 | F7DVJ0_MONDO | | Opsin 5 | OPN5 |
| A0A2K5EFU2 | A0A2K5EFU2_AOTNA | | Opsin 5 | OPN5 |
| A0A5F8H1F1 | A0A5F8H1F1_MONDO | | Opsin 5 | OPN5 |
| A0A2Y9H826 | A0A2Y9H826_NEOSC | | opsin-5 | OPN5 |
| G3W284 | G3W284_SARHA | | Opsin 5 | OPN5 |
| A0A3Q0CTY5 | A0A3Q0CTY5_MESAU | | opsin-5 | Opn5 |
| A0A6P5NS60 | A0A6P5NS60_MUSCR | | opsin-5 | Opn5 |
| H0V671 | H0V671_CAVPO | | Opsin 5 | OPN5 |
| I3M1B1 | I3M1B1_ICTTR | | Opsin 5 | OPN5 |
| Q7TQN6 | Q7TQN6_RAT | | G protein-coupled receptor 136 | Opn5 Gpr136 |
| | | | (Opsin 5) | |
| A0A287CZD4 | A0A287CZD4_ICTTR | | Opsin 5 | OPN5 |
| A0A1W6KZ83 | A0A1W6KZ83_9RODE | | Neuropsin | OPN5 |
| A0A6I9MCW1 | A0A6I9MCW1_PERMB | | opsin-5 | Opn5 |
| A0A6P3EVC3 | A0A6P3EVC3_OCTDE | | opsin-5 | Opn5 |
| A0A1S3FD42 | A0A1S3FD42_DIPOR | | LOW QUALITY PROTEIN: | Opn5 |
| | | | opsin-5 | |
| A0A6A4VE33 | A0A6A4VE33_AMPAM | | Opsin-5 | OPN5 FJT64_010458 |
| A0A4P2TKU6 | A0A4P2TKU6_PAROL | | Neuropsin | OPN5 |
| A0A670IDE8 | A0A670IDE8_PODMU | | G_PROTEIN_RECEP_F1_2 | OPN5 |
| | | | domain-containing protein | |
| A0A1U7S163 | A0A1U7S163_ALLSI | | opsin-5 | OPN5 |
| A0A670Y2N7 | A0A670Y2N7_PSETE | | Opsin 5 | OPN5 |
| K7FFW2 | K7FFW2_PELSI | | G_PROTEIN_RECEP_F1_2 | OPN5 |
| | | | domain-containing protein | |
| D9N3D0 | D9N3D0_COTJA | | Opsin 5 | OPN5 |
| Q6VZZ7 | OPN5_MOUSE | reviewed | Opsin-5 (G-protein coupled | Opn5 Gpr136 Pgr12 |
| | | | receptor 136) (G-protein | |
| | | | coupled receptor PGR12) | |
| | | | (Neuropsin) | |
| D8KWH6 | D8KWH6_ZONAL | | Opsin 5 | OPN5 |
| A0A674PPK4 | A0A674PPK4_TAKRU | | G_PROTEIN_RECEP_F1_2 | opn5 |
| | | | domain-containing protein | |
| A0A674HDZ6 | A0A674HDZ6_TAEGU | | G_PROTEIN_RECEP_F1_2 | OPN5 |
| | | | domain-containing protein | |
| H2V568 | H2V568_TAKRU | | G_PROTEIN_RECEP_F1_2 | opn5 |
| | | | domain-containing protein | |
| A0A6J0H1N3 | A0A6J0H1N3_9PASS | | opsin-5 | OPN5 |
| A0A672UEH1 | A0A672UEH1_STRHB | | Opsin 5 | OPN5 |
| A0A672UBX7 | A0A672UBX7_STRHB | | Opsin 5 | OPN5 |
| A0A6J0U919 | A0A6J0U919_9SAUR | | opsin-5 | OPN5 |
| A0A6J8E395 | A0A6J8E395_MYTCO | | OPN5 | MCOR_46347 |
| A0A6J7ZZ06 | A0A6J7ZZ06_MYTCO | | OPN5 | MCOR_1439 |
| A0A2J8RKQ7 | A0A2J8RKQ7_PONAB | | OPN5 isoform 1 | CR201_G0050220 |
| A0A2J8P0V4 | A0A2J8P0V4_PANTR | | OPN5 isoform 4 | CK820_G0007353 |
| A0A212D584 | A0A212D584_CEREH | | OPN5 | Celaphus_00014381 |

| Entry | Organism | Length |
|---|---|---|
| E0R7P4 | Xenopus laevis (African clawed frog) | 341 |
| A0A455SGG5 | Ambigolimax valentianus | 425 |
| A0A4Z2FX25 | Liparis tanakae (Tanaka's snailfish) | 178 |
| A0A4Z2FH27 | Liparis tanakae (Tanaka's snailfish) | 399 |
| A0A4Z2IDU8 | Liparis tanakae (Tanaka's snailfish) | 396 |
| A0A4Z2H0H0 | Liparis tanakae (Tanaka's snailfish) | 153 |
| A0A218USZ0 | Lonchura striata domestica (Bengalese finch) | 348 |
| A0A4Z2FVH4 | Liparis tanakae (Tanaka's snailfish) | 338 |
| A0A4Z2HA58 | Liparis tanakae (Tanaka's snailfish) | 311 |
| A0A218UGP1 | Lonchura striata domestica (Bengalese finch) | 417 |
| G1L3V2 | Ailuropoda melanoleuca (Giant panda) | 381 |
| A0A6P4X9I3 | Panthera pardus (Leopard) (Felis pardus) | 353 |
| A0A1S2ZDX4 | Erinaceus europaeus (Western European hedgehog) | 353 |
| A0A2I4C032 | Austrofundulus limnaeus | 353 |
| U3JFW4 | Ficedula albicollis (Collared flycatcher) (Muscicapa albicollis) | 357 |
| A0A2Y9NPU7 | Delphinapterus leucas (Beluga whale) | 362 |
| A0A1U7U6G6 | Carlito syrichta (Philippine tarsier) (Tarsius syrichta) | 354 |
| A0A6I9I544 | Vicugna pacos (Alpaca) (Lama pacos) | 353 |
| M3YLS7 | Mustela putorius furo (European domestic ferret) (Mustela furo) | 377 |
| A0A5F9CCV1 | Oryctolagus cuniculus (Rabbit) | 366 |
| A0A671EF51 | Rhinolophus ferrumequinum (Greater horseshoe bat) | 380 |
| A0A6P6I1D4 | Puma concolor (Mountain lion) | 353 |
| G3RKG7 | Gorilla gorilla gorilla (Western lowland gorilla) | 382 |
| G1NYV5 | Myotis lucifugus (Little brown bat) | 353 |
| A0A6J2L9P4 | Phyllostomus discolor (pale spear-nosed bat) | 353 |
| A0A2K6AXI4 | Macaca nemestrina (Pig-tailed macaque) | 354 |
| A0A6J3JM90 | Sapajus apella (Brown-capped capuchin) (Cebus apella) | 354 |
| A0A452TE17 | Ursus maritimus (Polar bear) (Thalarctos maritimus) | 361 |
| A0A384C5D1 | Ursus maritimus (Polar bear) (Thalarctos maritimus) | 353 |
| A0A2K5U4B7 | Macaca fascicularis (Crab-eating macaque) (Cynomolgus | 354 |
| | monkey) | |
| A0A2I3MZV4 | Papio anubis (Olive baboon) | 354 |
| A0A2K5U4B3 | Macaca fascicularis (Crab-eating macaque) (Cynomolgus | 382 |
| | monkey) | |
| Q6U736 | Homo sapiens (Human) | 354 |
| F6UZB2 | Xenopus tropicalis (Western clawed frog) (Silurana tropicalis) | 345 |
| A0A4W3IAF8 | Callorhinchus milii (Ghost shark) | 340 |
| A0A6P7HHM6 | Parambassis ranga (Indian glassy fish) | 355 |
| H3B1A3 | Latimeria chalumnae (Coelacanth) | 290 |
| A0A4W3I3H5 | Callorhinchus milii (Ghost shark) | 333 |
| A0A1S3MCD5 | Salmo salar (Atlantic salmon) | 328 |
| A0A4W4FPG5 | Electrophorus electricus (Electric eel) (Gymnotus electricus) | 333 |
| A0A6P7LVJ1 | Betta splendens (Siamese fighting fish) | 308 |
| A0A6P7LVE3 | Betta splendens (Siamese fighting fish) | 365 |
| A0A674IKC9 | Terrapene carolina triunguis (Three-toed box turtle) | 372 |
| A0A674IMF3 | Terrapene carolina triunguis (Three-toed box turtle) | 347 |
| F1NEY2 | Gallus gallus (Chicken) | 357 |
| E6P6L8 | Danio rerio (Zebrafish) (Brachydanio rerio) | 352 |
| A0A671TVX9 | Sparus aurata (Gilthead sea bream) | 357 |
| A0A7M4FP40 | Crocodylus porosus (Saltwater crocodile) (Estuarine crocodile) | 357 |
| A0A671TVX4 | Sparus aurata (Gilthead sea bream) | 353 |
| A0A6I9Y3G3 | Thamnophis sirtalis | 277 |
| G1KNV3 | Anolis carolinensis (Green anole) (American chameleon) | 347 |
| A0A493T549 | Anas platyrhynchos platyrhynchos (Northern mallard) | 337 |
| A0A6I9HELA | Geospiza fortis (Medium ground-finch) | 354 |
| A0A218UPZ6 | Lonchura striata domestica (Bengalese finch) | 304 |
| D8KW68 | Zonotrichia albicollis (White-throated sparrow) | 354 |
| A0A663EIX5 | Aquila chrysaetos chrysaetos | 343 |
| G1NNA7 | Meleagris gallopavo (Wild turkey) | 358 |
| A0A663EK31 | Aquila chrysaetos chrysaetos | 370 |
| A0A6J0Z1K0 | Odocoileus virginianus texanus | 353 |
| A0A6P3J431 | Bison bison bison | 353 |
| A0A2K5R3Y3 | Cebus imitator (Panamanian white-faced capuchin) (Cebus | 382 |
| | capucinus imitator) | |
| A0A671EF86 | Rhinolophus ferrumequinum (Greater horseshoe bat) | 354 |
| A0A6I9ZSG3 | Acinonyx jubatus (Cheetah) | 353 |
| A0A2K6R8Q2 | Rhinopithecus roxellana (Golden snub-nosed monkey) | 354 |
| | (Pygathrix roxellana) | |
| A0A4W2GZA3 | Bos indicus × Bos taurus (Hybrid cattle) | 355 |
| U3J4Q3 | Anas platyrhynchos platyrhynchos (Northern mallard) | 380 |
| A0A6J2V8J5 | Chanos chanos (Milkfish) (Mugil chanos) | 355 |
| A0A493T6P1 | Anas platyrhynchos platyrhynchos (Northern mallard) | 400 |
| A0A6J2J0L1 | Pipra filicauda (Wire-tailed manakin) | 354 |
| A0A6P9CE92 | Pantherophis guttatus (Corn snake) (Elaphe guttata) | 357 |
| A0A6J1V4P8 | Notechis scutatus (mainland tiger snake) | 357 |
| A0A288HLV3 | Anser cygnoid (Swan goose) | 355 |
| A0A151PID4 | Alligator mississippiensis (American alligator) | 369 |
| Q5RIV6 | Danio rerio (Zebrafish) (Brachydanio rerio) | 352 |
| D6RDV4 | Homo sapiens (Human) | 382 |
| J3KPQ2 | Homo sapiens (Human) | 353 |
| F6XNY7 | Ornithorhynchus anatinus (Duckbill platypus) | 327 |
| A0A2K6FXK2 | Propithecus coquereli (Coquerel's sifaka) (Propithecus verreauxi | 354 |
| | coquereli) | |
| E2RPZ0 | Canis lupus familiaris (Dog) (Canis familiaris) | 380 |
| A0A2K6V732 | Saimiri boliviensis boliviensis (Bolivian squirrel monkey) | 381 |
| A0A4X2K722 | Vombatus ursinus (Common wombat) | 353 |
| A0A6P5KYE6 | Phascolarctos cinereus (Koala) | 355 |
| A0A2K6FXJ4 | Propithecus coquereli (Coquerel's sifaka) (Propithecus verreauxi | 380 |
| | coquereli) | |
| A0A4X2JZA4 | Vombatus ursinus (Common wombat) | 353 |
| G1SX53 | Oryctolagus cuniculus (Rabbit) | 353 |
| A0A2U3WI94 | Odobenus rosmarus divergens (Pacific walrus) | 353 |
| A0A2K6V724 | Saimiri boliviensis boliviensis (Bolivian squirrel monkey) | 354 |
| A0A3Q7XKC9 | Ursus arctos horribilis | 353 |
| A0A452RBH1 | Ursus americanus (American black bear) (Euarctos americanus) | 353 |
| G1QVY1 | Nomascus leucogenys (Northern white-cheeked gibbon) | 382 |
| | (Hylobates leucogenys) | |
| G1QVX6 | Nomascus leucogenys (Northern white-cheeked gibbon) | 354 |
| | (Hylobates leucogenys) | |
| G3SJY5 | Gorilla gorilla gorilla (Western lowland gorilla) | 354 |
| A0A7N9CSX2 | Macaca fascicularis (Crab-eating macaque) (Cynomolgus | 353 |
| | monkey) | |
| A0A384B2Q9 | Balaenoptera acutorostrata scammoni (North Pacific minke | 353 |
| | whale) (Balaenoptera davidsoni) | |
| A0A2K6L978 | Rhinopithecus bieti (Black snub-nosed monkey) (Pygathrix | 333 |
| | bieti) | |
| A0A2K6AXE7 | Macaca nemestrina (Pig-tailed macaque) | 382 |
| A0A2J8P0S9 | Pan troglodytes (Chimpanzee) | 354 |
| W5PR22 | Ovis aries (Sheep) | 377 |
| F7DJ88 | Callithrix jacchus (White-tufted-ear marmoset) | 382 |
| A0A2K5R3Z8 | Cebus imitator (Panamanian white-faced capuchin) (Cebus | 354 |
| | capucinus imitator) | |
| F6PHB6 | Callithrix jacchus (White-tufted-ear marmoset) | 354 |
| M3WMC9 | Felis catus (Cat) (Felis silvestris catus) | 353 |
| A0A2K5L5D5 | Cercocebus atys (Sooty mangabey) (Cercocebus torquatus atys) | 382 |
| E1BNN4 | Bos taurus (Bovine) | 353 |
| F6RFW7 | Macaca mulatta (Rhesus macaque) | 354 |
| A0A2J8RKP9 | Pongo abelii (Sumatran orangutan) (Pongo pygmaeus abelii) | 354 |
| A0A3Q7RXX8 | Vulpes vulpes (Red fox) | 353 |
| A0A2K5L5D9 | Cercocebus atys (Sooty mangabey) (Cercocebus torquatus atys) | 354 |
| H0WJY2 | Otolemur garnettii (Small-eared galago) (Garnett's greater | 352 |
| | bushbaby) | |
| A0A6P3ENQ6 | Ovis aries (Sheep) | 353 |
| G3UA68 | Loxodonta africana (African elephant) | 377 |
| A0A6P5DVT1 | Bos indicus (Zebu) | 360 |
| A0A0D9RJS4 | Chlorocebus sabaeus (Green monkey) (Cercopithecus sabaeus) | 352 |
| I3LTK7 | Sus scrofa (Pig) | 378 |
| A0A2K5Z564 | Mandrillus leucophaeus (Drill) (Papio leucophaeus) | 382 |
| A0A5G2R7I1 | Sus scrofa (Pig) | 353 |
| A0A6I9JGH7 | Chrysochloris asiatica (Cape golden mole) | 353 |
| A0A2K5Z517 | Mandrillus leucophaeus (Drill) (Papio leucophaeus) | 354 |
| A0A452FM79 | Capra hircus (Goat) | 353 |
| F6SJH5 | Equus caballus (Horse) | 382 |
| A0A2R9BTW5 | Pan paniscus (Pygmy chimpanzee) (Bonobo) | 353 |
| A0A2Y9FNI2 | Physeter macrocephalus (Sperm whale) (Physeter catodon) | 353 |
| A0A340WR35 | Lipotes vexillifer (Yangtze river dolphin) | 353 |
| A0A6J2DJL3 | Zalophus californianus (California sealion) | 353 |
| A0A4X1UZM3 | Sus scrofa (Pig) | 378 |
| A0A673TX31 | Suricata suricatta (Meerkat) | 381 |
| A0A341D5X7 | Neophocaena asiaeorientalis asiaeorientalis (Yangtze finless | 353 |
| | porpoise) (Neophocaena phocaenoides subsp. asiaeorientalis) | |
| A0A667FWA1 | Lynx canadensis (Canada lynx) | 376 |
| A0A5B7H9S7 | Portunus trituberculatus (Swimming crab) (Neptunus | 74 |
| | trituberculatus) | |
| A0A337SC50 | Felis catus (Cat) (Felis silvestris catus) | 376 |
| H2RD19 | Pan troglodytes (Chimpanzee) | 382 |
| A0A2U3X849 | Leptonychotes weddellii (Weddell seal) (Otaria weddellii) | 365 |
| G3THK6 | Loxodonta africana (African elephant) | 360 |
| A0A2U3V1E1 | Tursiops truncatus (Atlantic bottle-nosed dolphin) (Delphinus | 353 |
| | truncatus) | |
| A0A096NIY4 | Papio anubis (Olive baboon) | 382 |
| A0A6P3PSZ2 | Pteropus vampyrus (Large flying fox) | 353 |
| A0A2K5EFR2 | Aotus nancymaae (Ma's night monkey) | 382 |
| A0A3Q7QKC2 | Callorhinus ursinus (Northern fur seal) | 353 |
| F7DVJ0 | Monodelphis domestica (Gray short-tailed opossum) | 346 |
| A0A2K5EFU2 | Aotus nancymaae (Ma's night monkey) | 354 |
| A0A5F8H1F1 | Monodelphis domestica (Gray short-tailed opossum) | 347 |
| A0A2Y9H826 | Neomonachus schauinslandi (Hawaiian monk seal) (Monachus | 353 |
| | schauinslandi) | |
| G3W284 | Sarcophilus harrisii (Tasmanian devil) (Sarcophilus laniarius) | 355 |
| A0A3Q0CTY5 | Mesocricetus auratus (Golden hamster) | 254 |
| A0A6P5NS60 | Mus caroli (Ryukyu mouse) (Ricefield mouse) | 377 |
| H0V671 | Cavia porcellus (Guinea pig) | 333 |
| I3M1B1 | Ictidomys tridecemlineatus (Thirteen-lined ground squirrel) | 353 |
| | (Spermophilus tridecemlineatus) | |
| Q7TQN6 | Rattus norvegicus (Rat) | 534 |
| A0A287CZD4 | Ictidomys tridecemlineatus (Thirteen-lined ground squirrel) | 378 |
| | (Spermophilus tridecemlineatus) | |
| A0A1W6KZ83 | Cricetulus barabensis (striped dwarf hamster) | 377 |
| A0A6I9MCW1 | Peromyscus maniculatus bairdii (Prairie deer mouse) | 377 |
| A0A6P3EVC3 | Octodon degus (Degu) (Sciurus degus) | 353 |
| A0A1S3FD42 | Dipodomys ordii (Ord's kangaroo rat) | 603 |
| A0A6A4VE33 | Amphibalanus amphitrite (Striped barnacle) (Balanus | 358 |
| | amphitrite) | |
| A0A4P2TKU6 | Paralichthys olivaceus (Bastard halibut) (Hippoglossus | 354 |
| | olivaceus) | |
| A0A670IDE8 | Podarcis muralis (Wall lizard) (Lacerta muralis) | 358 |
| A0A1U7S163 | Alligator sinensis (Chinese alligator) | 350 |
| A0A670Y2N7 | Pseudonaja textilis (Eastern brown snake) | 385 |
| K7FFW2 | Pelodiscus sinensis (Chinese softshell turtle) (Trionyx sinensis) | 369 |
| D9N3D0 | Coturnix japonica (Japanese quail) (Coturnix coturnix japonica) | 378 |
| Q6VZZ7 | Mus musculus (Mouse) | 377 |
| D8KWH6 | Zonotrichia albicollis (White-throated sparrow) | 354 |
| A0A674PPK4 | Takifugu rubripes (Japanese pufferfish) (Fugu rubripes) | 363 |
| A0A674HDZ6 | Taeniopygia guttata (Zebra finch) (Poephila guttata) | 354 |
| H2V568 | Takifugu rubripes (Japanese pufferfish) (Fugu rubripes) | 394 |
| A0A6J0H1N3 | Lepidothrix coronata (blue-crowned manakin) | 351 |
| A0A672UEH1 | Strigops habroptila (Kakapo) | 377 |
| A0A672UBX7 | Strigops habroptila (Kakapo) | 349 |
| A0A6J0U919 | Pogona vitticeps (central bearded dragon) | 348 |
| A0A6J8E395 | Mytilus coruscus (Sea mussel) | 317 |
| A0A6J7ZZ06 | Mytilus coruscus (Sea mussel) | 235 |
| A0A2J8RKQ7 | Pongo abelii (Sumatran orangutan) (Pongo pygmaeus abelii) | 382 |
| A0A2J8P0V4 | Pan troglodytes (Chimpanzee) | 353 |
| A0A212D584 | Cervus elaphus hippelaphus (European red deer) | 263 |

### Example 5

### Animals:

8-16 weeks rd1/rd1 retinitis pigmentosa (RP) model mice, which were fed on a 12/12 light/dark cycle (lights off at 8 μm).

### Construction of AAV Vector:

The plasmids needed to package AAV virus, include pAAV-mSNCG-chicken opn5m-t2a-EGFP, pAAV-mSNCG-chicken opn5m-t2a-mcherry, pAAV-mSNCG-chicken opn5m, and pAAV-mSNCG-EGFP.

### Packaging and Production of Adeno-Associated Virus (AAV):

Recombinant AAV was prepared by co-transfection of plasmids. AAV2.7M8 and AAV2/8subtypes were packaged, respectively. Both of them include mSNCG-chicken opn5m-t2a-EGFP, mSNCG-chicken opn5m-t2a-mcherry, mSNCG-chicken opn5m and mSNCG-EGFP.

Intraocular Injection of AAV into Mice:

After anesthesia, mice were injected with 1l AAV into the vitreous cavity after passing through the sclera with ultra-fine glass electrode, and the electrode was pulled out after several seconds. Follow up experiments were conducted 4 weeks after AAV injection.

### Immunofluorescence:

In order to confirm whether AAV successfully infects retinal cells and compare the infection efficiency and virus specificity among various AAV subtypes, the immunofluorescence experiment is needed. After 4 weeks of AAV injection, the mouse retina was taken out and fixed in 4% paraformaldehyde for 30 minutes. The fixed and cleaned retina was embedded, and was sliced vertically with Leica cryomicrotome, with a thickness of 15 μm. The slices were washed with PBS, then sealed with 3% BSA (bovine serum albumin) at room temperature for 1 hour. Then the first anti-EGFP antibody is diluted with 3% BSA with 1:500, and incubated at 4° C. for 48 hours. After cleaning the first antibody, incubating it with the fluorescent labeled second antibody for 2 hours, pasting the stained retinal slice on the glass slide, and confocal scanning to obtain the fluorescence image after sealing. Analyzing and comparing the infection efficiency of each AAV to retinal ganglion cell (RGC), and the fluorescence intensity of EGFP, and select the AAV subtypes with high infection rate and good specificity for the next experiment.

### Electrophysiological Test:

In order to further confirm whether cOPN5 maintains its physiological activity in RGC cells after successful expression of the AAV, electrophysiological experiments are needs. The AAVs having high infection rate and good specificity were injected into the eyes of rd1/rd1 (purchased from GemPharmatech Co., Ltd) mice. After 4 weeks of virus injection, the mouse retina was taken out and the retinal slice was placed in the electrophysiological recording chamber. The RGC layer of the retina was upward. In order to prevent light damage to the retina, the laser was turned off after the somatic cells expressing GFP were identified by the fluorescence microscope. The current intensity was recorded after cells were stimulated by 488 nm laser with different light intensity.

### Behavior Test:

The visual receptor cells of RD1/rd1 mice have degenerated. To verify whether visual information can be transmitted to the brain through infected ganglion cells, so as to restore their lost visual function, we selected several visual function tests:

### (1) Pupillary Light Reflex (PLR)

In Rd1/rd1 mice, the pupil can only respond to strong light. PLR experiment was conducted 4 weeks after injection of AAV into eyes of mice. Different intensity of light is utilized to stimulate the pupil of cOPN5 expressing mice and EGFP expressing mice to record the change degree of the pupil, and evaluate the sensitivity of mice to light through the change degree of the pupil.

### (2) Open Field Avoidance Test

Normal mice will avoid open and bright spaces. This innate tendency is the basis for a simple test of their visual ability. In the experiment, the mice were placed in a lighted space, and there was also a dark shelter. The visual ability of mice was evaluated by measuring the proportion of time they spent.

### Safety Test:

Long term heterologous expression of genes will have different effects on expressed tissues. Long term experiments are needed to evaluate the safety of heterologous expression, and test whether heterologous expression genes will be stably expressed in tissues for a long time. AAV was injected into the eyes for 6 months, and the above immunofluorescence, electrophysiological test and behavioral test were repeated one year later to detect the expression level of cOPN5, and whether the physiological activity changed due to long-term expression, and detect whether there is inflammatory reaction in retinal tissue.

### Results:

As shown in FIG. 11, A showed expression of cOPN5 protein in retinal ganglion cells in the rd1/rd1 mouse;
B shows microglia marker Iba1 staining of retinal slices after injection. H₂O₂-injected mice (positive control) showed strong activation of microglia. Few basal Iba1 signals were observed in the AAV-cOPN5-t2a-EGFP injected retina after 1 month injection, similar to that observed in AAV-EGFP-injected retina, AAV-cOPN5-t2a-EGFP injected retina after 10 month injection and no injection retinal. Red, Iba1; green, cOPN5 or EGFP; blue, DAPI (4′,6-diamidino-2-phenylindole) signal indicating cell nuclei. Scale bar, 50 μm;
C shows RGC marker brn3a staining of retinal slices. Red, brn3a; green, cOPN5; blue, signal indicating cell nuclei. Scale bar, 50 μm.

D shows Fundus fluorescence imaging.

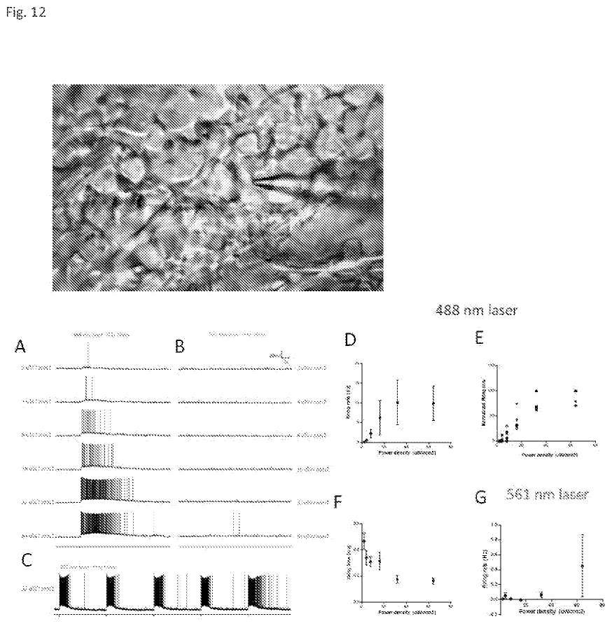

As shown in FIG. 12, A shows representative responses of RGC from C3H mice injected AAV-Copn5-t2a-EGFP during different power 488 nm laser stimulation;
B shows representative responses of RGC from C3H mice injected AAV-Copn5-t2a-EGFP during different power 561 nm laser stimulation;
C shows raw trace that cOpn5 mediated reliable and reproducible photoactivation of RGC;
D and E Group data show the RGC firing rates after different power 488 nm laser stimulation, (n=6);
F Group data show the delay time after different power 488 nm laser stimulation. (n=6)

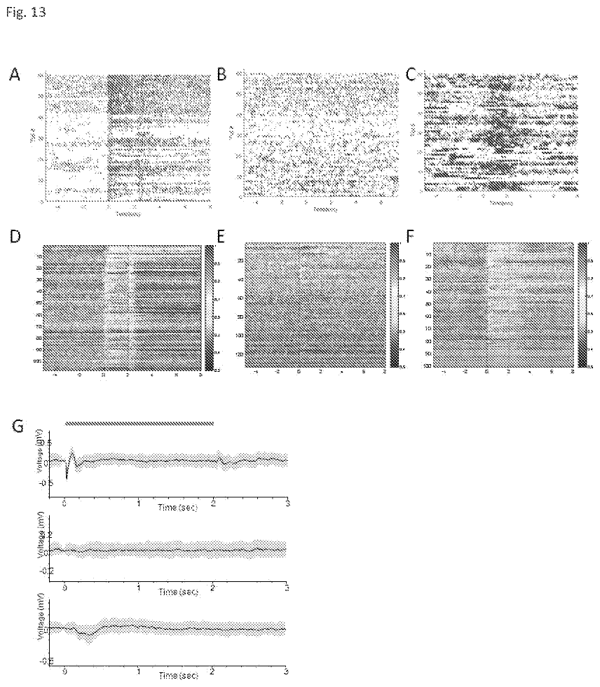

As shown in FIG. 13, A shows representative responses of v1 neurons from C57 mice during 2s 200 lux light stimulation;
B shows representative responses of vi neurons from C3H mice injected AAV-EGFP during 2s 200 lux light stimulation;
C shows representative responses of vi neurons from C3H mice injected AAV-cOPN5-t2a-EGFP during 2s 200 lux light stimulation;
D shows heat maps indicating the ROC representation of the peristimulus time histogram data from the C57 mice vi neurons that were tested 2s 200 lux light stimulation. (n=107);
E shows heat maps indicating the ROC representation of the peristimulus time histogram data from the C3H mice injected AAV-EGFP vi neurons that were tested 2s 200 lux light stimulation. (n=133);
F shows heat maps indicating the ROC representation of the peristimulus time histogram data from the C3H mice injected AAV-cOPN5-t2a-EGFP v1 neurons that were tested 2s 200 lux light stimulation. (n=100);
G shows visually evoked potentials (VEPs) of C57(top), AAV-EGFP injected rd/rd mice (middle), and AAV-cOPN5-EGFP injected rd1/rd1 under 2s light illumination. (n=6).

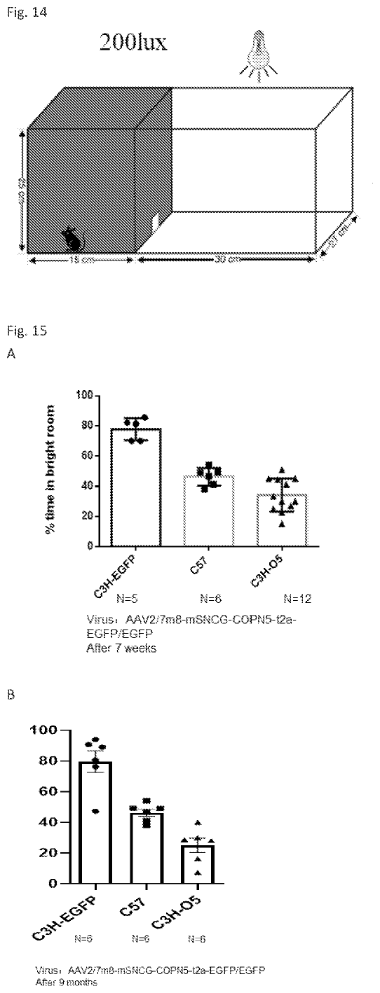

### FIG. 14 Schematically Shows Open Field Avoidance Test:

Method: The light/dark box (45×27×25 cm) was made of Plexiglas and consisted of two chambers connected by an opening (4×5 cm) located at floor level in the center of the dividing wall. The light box occupies about ⅔ of the whole light/dark box, and the dark box occupy about ⅓ of the whole light/dark box. The test field was diffusely illuminated at 200 lux. Mice were carried into the testing room in their home cage. A trial began when the mouse was placed inside the dark shelter for a 2-min habituation period, with the opening from dark to light spaces closed. The mouse was then allowed to leave the shelter and explore the illuminated field for 5 min. For each mouse, the length of time the animal spent in the light side of the box was recorded. A video camcorder located above the center of the box provided a permanent record of the behavior of the mouse. Mice were then removed from the box and returned to the home cage.

The results of the open field avoidance test were shown in FIG. 15, wherein FIG. 15A shows that after 7 weeks, the blind (rd/rd) mice spent about 80% time in the light box, and the control mice (normal mice) spent about 50% time in the light box, and the AAV-EGFP injected rd1/rd1 mice spent about 30% time in the light box; and

FIG. 15B shows that after 9 months, the blind (rd/rd) mice spent about 80% time in the light box, and the control mice (normal mice) spent about 50% time in the light box, and the AAV-EGFP injected rd1/rd1 mice spent about 20% time in the light box.

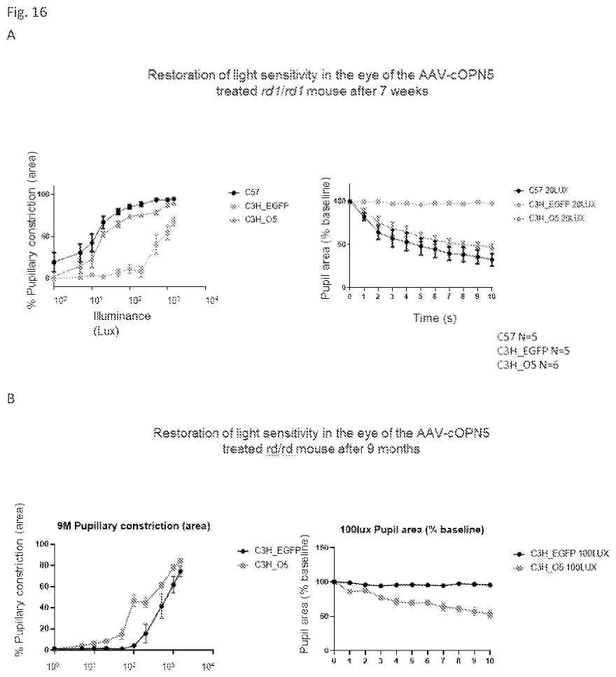

FIG. 16 shows the restoration of light sensitivity in the eye of the AAV-cOPN5 treated rd1/rd1 mice after 7 weeks (A) and 9 months (B) respectively. It found that AAV-cOPN5 treated rd1/rd1 mice (C3H_O5) have similar % pupillary constriction (area) to the normal mice (C57), and the rd1/rd1 mice (C3H_EGFP) shows almost no % pupillary constriction (area).

### REFERENCES

1 Hauser, A. S., Attwood, M. M., Rask-Andersen, M., Schioth, H. B. & Gloriam, D. E. Trends in GPCR drug discovery: new agents, targets and indications. Nat Rev Drug Discov 16, 829-842, doi:10.1038/nrd.2017.178 (2017).
2 Wettschureck, N. & Offermanns, S. Mammalian G proteins and their cell type specific functions. Physiol Rev 85, 1159-1204, doi:10.1152/physrev.00003.2005 (2005).
3 Exton, J. H. Regulation of phosphoinositide phospholipases by hormones, neurotransmitters, and other agonists linked to G proteins. Annu Rev Pharmacol Toxicol 36, 481-509, doi:10.1146/annurev.pa.36.040196.002405 (1996).
4 Ritter, S. L. & Hall, R. A. Fine-tuning of GPCR activity by receptor-interacting proteins. Nature reviews Molecular cell biology 10, 819-830 (2009).
5 Kadamur, G. & Ross, E. M. Mammalian phospholipase C. Annu Rev Physiol 75, 127-154, doi:10.1146/annurev-physiol-030212-183750 (2013).
6 Gomez, J. L. et al. Chemogenetics revealed: DREADD occupancy and activation via converted clozapine. Science 357, 503-507, doi:10.1126/science.aan2475 (2017).
7 Adams, S. R. & Tsien, R. Y. Controlling cell chemistry with caged compounds. Annual review of physiology 55, 755-784 (1993).
8 Boyden, E. S., Zhang, F., Bamberg, E., Nagel, G. & Deisseroth, K. Millisecond-timescale, genetically targeted optical control of neural activity. Nat Neurosci 8, 1263-1268, doi:10.1038/nn1525 (2005).
9 Fenno, L., Yizhar, O. & Deisseroth, K. The Development and Application of Optogenetics. Annual Review of Neuroscience 34, 389-412, doi:10.1146/annurev-neuro-061010-113817 (2011).
10 Quadrato, G. et al. Cell diversity and network dynamics in photosensitive human brain organoids. Nature 545, 48-53 (2017).
11 Rost, B. R., Schneider-Warme, F., Schmitz, D. & Hegemann, P. Optogenetic tools for subcellular applications in neuroscience. Neuron 96, 572-603 (2017).
12 Tye, K. M. & Deisseroth, K. Optogenetic investigation of neural circuits underlying brain disease in animal models. Nature Reviews Neuroscience 13, 251-266 (2012).
13 Zhang, F. et al. The microbial opsin family of optogenetic tools. Cell 147, 1446-1457 (2011).
14 Airan, R. D., Thompson, K. R., Fenno, L. E., Bernstein, H. & Deisseroth, K. Temporally precise in vivo control of intracellular signalling. Nature 458, 1025-1029, doi:10.1038/nature07926 (2009).
15 Tichy, A. M., Gerrard, E. J., Sexton, P. M. & Janovjak, H. Light-activated chimeric GPCRs: limitations and opportunities. Curr Opin Struct Biol 57, 196-203, doi:10.1016/j.sbi.2019.05.006 (2019).
16 Koyanagi, M. & Terakita, A. Diversity of animal opsin-based pigments and their optogenetic potential. Biochim Biophys Acta 1837, 710-716, doi:10.1016/j.bbabio.2013.09.003 (2014).
17 Yau, K.-W. & Hardie, R. C. Phototransduction motifs and variations. Cell 139, 246-264 (2009).
18 Copits, B. A. et al. A photoswitchable GPCR-based opsin for presynaptic inhibition. Neuron 109, 1791-1809 e1711, doi:10.1016/j.neuron.2021.04.026 (2021).
19 Mahn, M. et al. Efficient optogenetic silencing of neurotransmitter release with a mosquito rhodopsin. Neuron 109, 1621-1635 e1628, doi:10.1016/j.neuron.2021.03.013 (2021).
20 Guler, A. D. et al. Melanopsin cells are the principal conduits for rod-cone input to non-image-forming vision. Nature 453, 102-105 (2008).
21 Hankins, M. W., Peirson, S. N. & Foster, R. G. Melanopsin: an exciting photopigment. Trends in neurosciences 31, 27-36 (2008).
22 Hattar, S., Liao, H.-W., Takao, M., Berson, D. M. & Yau, K.-W. Melanopsin-containing retinal ganglion cells: architecture, projections, and intrinsic photosensitivity. Science 295, 1065-1070 (2002).
23 Xue, T. et al. Melanopsin signalling in mammalian iris and retina. Nature 479, 67-73 (2011).
24 Qiu, X. et al. Induction of photosensitivity by heterologous expression of melanopsin. Nature 433, 745-749 (2005).
25 Melyan, Z., Tarttelin, E. E., Bellingham, J., Lucas, R. J. & Hankins, M. W. Addition of human melanopsin renders mammalian cells photoresponsive. Nature 433, 741-745, doi:10.1038/nature03344 (2005).
26 Kojima, D. et al. UV-sensitive photoreceptor protein OPNS in humans and mice. PLoS One 6, e26388, doi:10.1371/journal.pone.0026388 (2011).
27 Yamashita, T. et al. Opn5 is a UV-sensitive bistable pigment that couples with Gi subtype of G protein. Proc Natl Acad Sci USA 107, 22084-22089, doi:10.1073/pnas.1012498107 (2010).
28 Zhang, K. X. et al. Violet-light suppression of thermogenesis by opsin 5 hypothalamic neurons. Nature 585, 420-425, doi:10.1038/s41586-020-2683-0 (2020).
29 Nakane, Y. et al. A mammalian neural tissue opsin (Opsin 5) is a deep brain photoreceptor in birds. Proceedings of the National Academy of Sciences 107, 15264-15268 (2010).
30 Nakane, Y., Shimmura, T., Abe, H. & Yoshimura, T. Intrinsic photosensitivity of a deep brain photoreceptor. Current Biology 24, R596-R597 (2014).
31 Rios, M. N., Marchese, N. A. & Guido, M. E. Expression of Non-visual Opsins Opn3 and Opn5 in the Developing Inner Retinal Cells of Birds. Light-Responses in Muller Glial Cells. Front Cell Neurosci 13, 376, doi:10.3389/fncel.2019.00376 (2019).
32 Mederos, S. et al. Melanopsin for precise optogenetic activation of astrocyte-neuron networks. Glia 67, 915-934 (2019).
33 Taniguchi, M. et al. Structure of YM-254890, a Novel G_{q}/11 Inhibitor from Chromobacterium sp. QS3666. Tetrahedron 59, 4533-4538, doi:10.1016/s0040-4020(03)00680-x (2003).
34 Hartwig, J. et al. MARCKS is an actin filament crosslinking protein regulated by protein kinase C and calcium-calmodulin. Nature 356, 618-622 (1992).
35 Zhang, F., Wang, L.-P., Boyden, E. S. & Deisseroth, K. Channelrhodopsin-2 and optical control of excitable cells. Nature methods 3, 785-792 (2006).
36 Lin, J. Y. A user's guide to channelrhodopsin variants: features, limitations and future developments. Experimental physiology 96, 19-25 (2011).
37 Gomez, J. L. et al. Chemogenetics revealed: DREADD occupancy and activation via converted clozapine. Science 357, 503-507 (2017).
38 Krashes, M. J. et al. Rapid, reversible activation of AgRP neurons drives feeding behavior in mice. The Journal of clinical investigation 121, 1424-1428 (2011).
39 Rogan, S. C., Roth, B. L. & Morrow, A. L. Remote Control of Neuronal Signaling. Pharmacological Reviews 63, 291-315, doi:10.1124/pr.110.003020 (2011).
40 Charles, A. C., Merrill, J. E., Dirksen, E. R. & Sandersont, M. J. Intercellular signaling in glial cells: calcium waves and oscillations in response to mechanical stimulation and glutamate. Neuron 6, 983-992 (1991).
41 Cornell-Bell, A. H., Finkbeiner, S. M., Cooper, M. S. & Smith, S. J. Glutamate induces calcium waves in cultured astrocytes: long-range glial signaling. Science 247, 470-473 (1990).
42 Zhang, F., Wang, L. P., Boyden, E. S. & Deisseroth, K. Channelrhodopsin-2 and optical control of excitable cells. Nat Methods 3, 785-792, doi:10.1038/nmeth936 (2006).
43 Davalos, D. et al. ATP mediates rapid microglial response to local brain injury in vivo. Nature neuroscience 8, 752-758 (2005).
44 Zhang, J.-m. et al. ATP released by astrocytes mediates glutamatergic activity-dependent heterosynaptic suppression. Neuron 40, 971-982 (2003).
45 Zhang, Z. et al. Regulated ATP release from astrocytes through lysosome exocytosis. Nature cell biology 9, 945-953 (2007).
46 Wu, Z. & Li, Y. New frontiers in probing the dynamics of purinergic transmitters in vivo. Neuroscience research 152, 35-43 (2020).
47 Wu, Z. et al., doi:10.1101/2021.02.24.432680 (2021).
48 Lawlor, P. A., Bland, R. J., Mouravlev, A., Young, D. & During, M. J. Efficient gene delivery and selective transduction of glial cells in the mammalian brain by AAV serotypes isolated from nonhuman primates. Molecular therapy 17, 1692-1702 (2009).
49 Lee, Y., Messing, A., Su, M. & Brenner, M. GFAP promoter elements required for region-specific and astrocyte-specific expression. Glia 56, 481-493 (2008).
50 Bal-Price, A., Moneer, Z. & Brown, G. C. Nitric oxide induces rapid, calcium-dependent release of vesicular glutamate and ATP from cultured rat astrocytes. Glia 40, 312-323, doi:10.1002/glia.10124 (2002).
51 Murakami, K., Nakamura, Y. & Yoneda, Y. Potentiation by ATP of lipopolysaccharide-stimulated nitric oxide production in cultured astrocytes. Neuroscience 117, 37-42 (2003).
52 Lezmy, J. et al. Astrocyte Ca(2+)-evoked ATP release regulates myelinated axon excitability and conduction speed. Science 374, eabh2858, doi:10.1126/science.abh2858 (2021).
53 Tian, L. et al. Imaging neural activity in worms, flies and mice with improved GCaMP calcium indicators. Nature methods 6, 875-881 (2009).
54 Jennings, J. H., Rizzi, G., Stamatakis, A. M., Ung, R. L. & Stuber, G. D. The inhibitory circuit architecture of the lateral hypothalamus orchestrates feeding. Science 341, 1517-1521 (2013).
55 Stuber, G. D. & Wise, R. A. Lateral hypothalamic circuits for feeding and reward. Nat Neurosci 19, 198-205, doi:10.1038/nn.4220 (2016).
56 Li, Y. et al. Hypothalamic Circuits for Predation and Evasion. Neuron 97, 911-924 e915, doi:10.1016/j.neuron.2018.01.005 (2018).
57 Zhang, X. & van den Pol, A. N. Rapid binge-like eating and body weight gain driven by zona incerta GABA neuron activation. Science 356, 853-859, doi:10.1126/science.aam7100 (2017).
58 Tsukamoto, H. & Terakita, A. Diversity and functional properties of bistable pigments. Photochemical & Photobiological Sciences 9, 1435-1443 (2010).
59 Ellis-Davies, G. C. Caged compounds: photorelease technology for control of cellular chemistry and physiology. Nature methods 4, 619-628 (2007).
60 Adams, S. R. & Tsien, R. Y. Controlling cell chemistry with caged compounds. Annu Rev Physiol 55, 755-784, doi:10.1146/annurev.ph.55.030193.003543 (1993).
61 Chen, T.-W. et al. Ultrasensitive fluorescent proteins for imaging neuronal activity. Nature 499, 295-300 (2013).
62 Jing, M. et al. An optimized acetylcholine sensor for monitoring in vivo cholinergic activity. Nature methods 17, 1139-1146 (2020).
63 Sun, F. et al. A genetically encoded fluorescent sensor enables rapid and specific detection of dopamine in flies, fish, and mice. Cell 174, 481-496. e419 (2018).
64 Wan, J. et al. A genetically encoded sensor for measuring serotonin dynamics. Nature Neuroscience 24, 746-752 (2021).
65 Zhao, Y. et al. An expanded palette of genetically encoded Ca2+ indicators. Science 333, 1888-1891 (2011).
66 Hochbaum, D. R. et al. All-optical electrophysiology in mammalian neurons using engineered microbial rhodopsins. Nat Methods 11, 825-833, doi:10.1038/nmeth.3000 (2014).
67 Sahel, J.-A. et al. Partial recovery of visual function in a blind patient after optogenetic therapy. Nature Medicine, 1-7 (2021).

1. An isolated light-sensitive opsin for restoring sensitivity to light of the retinal cell through activating Gq signaling, which is an isolated opsin from an organism, its homologs, its orthologs, its paralogs, fragments or variants thereof having the activity of restoring sensitivity to light of the retinal cell through activating Gq signaling.

2. (canceled)

3. The isolated opsin of claim 1, which shares at least 70%, at least 80%, at least 90%, at least 95%, at least 96%, at least 97%, at least 98%, or at least 99% identity to a wild type opsin in the organism, its homologs, its orthologs, its paralogs, fragments or variants thereof, and has the activity of restoring sensitivity to light of the retinal cell through activating Gq signaling.

4. The isolated opsin of claim 1, which is an isolated opsin 5 (Opn5) from an animal, its homologs, its orthologs, its paralogs, fragments or variants thereof having the activity of restoring sensitivity to light of the retinal cell through activating Gq signaling preferably the isolated opsins shares at least 70%, at least 80%, at least 90%, at least 95%, at least 96%, at least 97%, at least 98%, or at least 99% identity to the wild type opsin 5 (Opn5) in the animal, its homologs, its orthologs, its paralogs, fragments or variants thereof, and has the activity of restoring sensitivity to light of the retinal cell through activating Gq signaling.

5. (canceled)

6. The isolated opsin of claim 1, wherein the organism is a vertebrate animal.

7. The isolated opsin of claim 6, wherein the vertebrate animal is an avian, a reptile, or a fish, an amphibian, or a mammal,
preferably, the animal is an avian, including but not limited to chicken, duck, goose, ostrich, emu, rhea, kiwi, cassowary, turkey, quail, chicken, falcon, eagle, hawk, pigeon, parakeet, cockatoo, macaw, parrot, perching bird (such as, song bird), jay, blackbird, finch, warbler and sparrow; or
preferably, the animal is a reptile including but not limited to lizard, snake, alligator, turtle, crocodile, and tortoise; or
preferably, the animal is a fish including but not limited to catfish, eels, sharks, and swordfish; or
preferably, the animal is an amphibian including but not limited to a toad, frog, newt, and salamander.

8. The isolated opsin of claim 4, wherein the isolated opsin 5 (Opn5) is an isolated wild type opsin 5 (Opn5) from a chicken, or fragments or variants thereof having the activity of restoring sensitivity to light of the retinal cell through activating Gq signaling; or
the isolated opsin 5 (Opn5) shares at least 70%, at least 80%, at least 90%, at least 95%, at least 96%, at least 97%, at least 98%, or at least 99% identity to the wild type opsin 5 (Opn5) from the chicken, and has the activity of restoring sensitivity to light of the retinal cell through activating Gq signaling.

9. The isolated opsin of claim 4, wherein the isolated opsin 5 (Opn5) is an isolated wild type opsin 5 (Opn5) from a turtle, or fragments or variants thereof having the activity of restoring sensitivity to light of the retinal cell through activating Gq signaling; or
the isolated opsin 5 (Opn5) shares at least 70%, at least 80%, at least 90%, at least 95%, at least 96%, at least 97%, at least 98%, or at least 99% identity to the wild type opsin 5 (Opn5) from the turtle, and has the activity of restoring sensitivity to light of the retinal cell through activating Gq signaling.

10. The isolated opsin of claim 4, wherein the isolated opsin 5 (Opn5) has the amino acid sequence shown by SEQ ID NO:1 (cOpn5), or fragments or variants thereof having the activity of restoring sensitivity to light of the retinal cell through activating Gq signaling; or
the isolated opsin 5 (Opn5) shares at least 70%, at least 80%, at least 90%, at least 95%, at least 96%, at least 97%, at least 98%, or at least 99% identity to the amino acid sequence shown by SEQ ID NO:1 (cOpn5), and has the activity of restoring sensitivity to light of the retinal cell through activating Gq signaling.

11. The isolated opsin of claim 4, wherein the isolated opsin 5 (Opn5) has the amino acid sequence shown by SEQ ID NO:2 (tOpn5), or fragments or variants thereof having the activity of restoring sensitivity to light of the retinal cell through activating Gq signaling; or
the isolated opsin 5 (Opn5) shares at least 70%, at least 80%, at least 90%, at least 95%, at least 96%, at least 97%, at least 98%, or at least 99% identity to the amino acid sequence shown by SEQ ID NO:2 (tOpn5), and has the activity of restoring sensitivity to light of the retinal cell through activating Gq signaling.

12. The isolated opsin of claim 1, wherein the light has a wavelength ranging range of 360 nm-520 nm, preferably, 450-500, more preferably, 460-480 nm, in particular, 470 nm.

13. The isolated opsin of claim 1, wherein the retinal cell is a photoreceptor cell, a retinal rod cell, a retinal cone cell, a retinal ganglion cell, a bipolar cell, a ganglion cell, a horizontal cell, a multipolar neuron, a Müller cell or an Amacrine cell, or is treated with Methylnitrosourea.

14. An isolated nucleic acid encoding the isolated opsin of claim 1.

15. A chimeric gene comprising the sequence of the isolated nucleic acid in claim 14, operably linked to suitable regulatory sequences;
preferably, further comprises a gene encoding a marker, for example, a fluorescent protein.

16. A vector comprising the isolated nucleic acid in claim 14, 15, preferably the vector is selected from a group consisting of a eukaryotic vector, a prokaryotic expression vector, a viral vector, or a yeast vector.

17. (canceled)

18. The vector of claim 16, which is a herpes virus simplex vector, a vaccinia virus vector, or an adenoviral vector, an adeno-associated viral vector, a retroviral vector, or an insect vector.

19. (canceled)

20. An isolated cell or a cell culture, comprising the isolated nucleic acid of claim 14.

21. (canceled)

22. A method of treating or preventing a disease or condition mediated by or involving loss sensitivity to light of the retinal cell through activating Gq signaling in a subject, comprising administering the isolated opsin of claim 1 to a subject in need thereof, preferably the method comprises a step of administrating an AAV vector expressing cOpn5 subretinally or intravitreally,
more preferably, the AAV vector further expresses a fluorescent protein.

23. The method of claim 22, wherein the disease or condition comprises diseases or conditions benefiting from restoring sensitivity to light of the retinal cell through activating Gq signaling,
preferably the disease or condition includes diseases or conditions benefiting from activating retinal cells, more preferably from a photoreceptor cell, a retinal rod cell, a retinal cone cell, a retinal ganglion cell, a bipolar cell, a ganglion cell, a horizontal cell, a multipolar neuron, a Müller cell or an Amacrine cell, or is treated with Methylnitrosourea,
more preferably, the disease or condition includes damage of the external layer of the retina, photoreceptor loss or degeneration, retinal degenerative disease, loss sensitivity to light, or loss light perception, loss of vision due to a deficit in light perception or sensitivity, and/or blindness.

24. (canceled)

25. (canceled)

26. The method of claim 22, wherein the disease or condition comprises diseases associated with degeneration and/or death of retinal ganglion cells (RGC).

27. (canceled)

28. The method of claim 22, wherein the method further comprises applying blue light having a wavelength range of 360 nm-550 nm, and/or applying two-photon activation using light having a wavelength ≥920 nm.

29. (canceled)
